(19) United States
(12) Patent Application Publication
(10) Pub. No.: US 2011/0104256 A1
(43) Pub. Date: May 5, 2011

(54) METHODS FOR TREATING OR PREVENTING COLORECTAL CANCER

(76) Inventors: Yaolin Wang, Edison, NJ (US); Yan Wang, Warren, NJ (US); Ming Liu, Fanwood, NJ (US); Walter Robert Bishop, Pompto Plains, NJ (US); Cynthia Seidel-Dugan, Mountainside, NJ (US)

(21) Appl. No.: 12/934,458

(22) PCT Filed: Mar. 23, 2009
(86) PCT No.: PCT/US09/37953

§ 371 Date: Dec. 17, 2010

Related U.S. Application Data
(60) Provisional application No. 61/039,197, filed on Mar. 25, 2008.

(51) Int. Cl. A61K 9/127; A61K 39/395; A61K 38/20; A61K 38/19; A61K 38/21; A61P 35/00; B82Y 5/00

(52) U.S. Cl. 424/450; 424/172.1; 424/143.1; 424/178.1; 424/136.1; 424/133.1; 424/131.1; 424/85.2; 424/85.1; 424/85.7; 977/795; 977/915

(57) ABSTRACT

The present invention provides, for example, methods for treating or preventing colorectal cancer with an anti-IGF1R antibody in association with sunitinib or a combination of leucovorin and 5-fluorouracil.

This application claims the benefit of U.S. provisional patent application No. 61/039,197, filed Mar. 25, 2008, which is herein incorporated by reference in its entirety.

### FIELD OF THE INVENTION

The field of the invention relates, generally, to methods of treating or preventing colorectal cancer by administering an anti-IGF1R antibody in association with another chemotherapeutic agent.

### BACKGROUND OF THE INVENTION

Colorectal cancer, or cancer of the colon and/or rectum, is the second leading cause of cancer-related deaths in the United States, and the third most common cancer overall. The American Cancer Society estimates that, each year, more than 50,000 Americans die from colorectal cancer and approximately 155,000 new cases are diagnosed, accounting for 15% of all types of tumor. Eighty to 90 million Americans (approximately 25% of the U.S. population) are considered at risk because of age or other factors. More women over the age of 75 die from colorectal cancer than from breast cancer. The 5-year survival rate remains at approximately 45%.

The occurrence of colorectal appears to be influenced by both inherited and lifestyle factors. Predisposing conditions for colorectal cancer include familial adenomatous polyposis (FAP), hereditary nonpolyposis colon cancer (HNPCC) (i.e., Lynch I Syndrome and Lynch II Syndrome), inflammatory bowel disease, including both chronic ulcerative colitis (UC) and Crohn's disease, other family cancer syndromes (e.g., Peutz-Jegher Syndromem and familial juvenile polyposis), and adenomatous polyps (e.g., sessile, tubular, villous or pendunculated). Other risk factors include high-meat, high-fat and low-fiber diets, cigarette smoking, a sedentary lifestyle, and obesity.

Colorectal cancer, particularly advanced colorectal cancer, has historically been difficult to treat using only standard therapeutic approaches. Often, standard therapies only demonstrate efficacy in a relatively small percentage of patients. Thus, there remains a need in the art for highly effective treatments of colorectal cancer.

### SUMMARY OF THE INVENTION

The present invention addresses the need in the art for colorectal cancer treatments, for example, by provision of highly effective combinations of chemotherapeutic agents for the treatment and prevention of colorectal cancer. The combinations includes an anti-IGF1R antibody or antigen-binding fragment thereof in association with sunitinib or in association with leucovorin and 5-fluorouracil.

The present invention comprises a method for treating or preventing colorectal cancer in a subject (e.g., a human) comprising administering a therapeutically effective amount an isolated antibody or antigen-binding fragment thereof (e.g., an isolated antibody such as a monoclonal antibody, a labeled antibody, a bivalent antibody, a polyclonal antibody, a bispecific antibody, a chimeric antibody, a recombinant antibody, an anti-idiotypic antibody, a humanized antibody or a bispecific antibody) comprising one or more members selected from the group consisting of: (a) CDR-L1, CDR-L2 and CDR-L3 of the variable region of light chain C, light chain D, light chain E or light chain F; or (b) CDR-H1, CDR-H2 and CDR-H3 of the variable region of heavy chain A or heavy chain b; or both; in association with leucovorin and 5-fluorouracil; or in association with sunitinib. In an embodiment of the invention, CDR-L1 comprises the amino acid sequence:

(SEQ ID NO: 1)
Arg Ala Ser Gln Ser Ile Gly Ser Ser Leu His;

CDR-L2 comprises the amino acid sequence:

| | |
|---|---|
| Tyr Ala Ser Gln Ser Leu Ser; | (SEQ ID NO: 2) |

CDR-L3 comprises the amino acid sequence:

| | |
|---|---|
| His Gln Ser Ser Arg Leu Pro His Thr; | (SEQ ID NO: 3) |

CDR-H1 comprises the amino acid sequence:

(SEQ ID NO: 4)
Ser Phe Ala Met His
or
(SEQ ID NO: 5)
Gly Phe Thr Phe Ser Ser Phe Ala Met His;

CDR-H2 comprises the amino acid sequence:

(SEQ ID NO: 6)
Val Ile Asp Thr Arg Gly Ala Thr Tyr Tyr Ala Asp
Ser Val Lys Gly

and
CDR-H3 comprises the amino acid sequence:

(SEQ ID NO: 7)
Leu Gly Asn Phe Tyr Tyr Gly Met Asp Val.

In an embodiment of the invention, the antibody or fragment comprises a light chain variable region comprising amino acids 20-128 of SEQ ID NO: 9, 11, 13 or 15 and a heavy chain variable region comprising amino acids 20-137 of SEQ ID NO: 17 or 19. In an embodiment of the invention, the antibody or fragment is a fragment and the fragment is a camelized single domain antibody, a diabody, an scfv, an scfv dimer, a dsfv, a (dsfv)₂, a dsFv-dsfv′, a bispecific ds diabody, an Fv, an Fab, an Fab′, an F(ab′)₂, or a domain antibody. In an embodiment of the invention, the antibody or fragment is linked to a constant region such as a κ light chain, γ1 heavy chain, γ2 heavy chain, γ3 heavy chain or γ4 heavy chain. In an embodiment of the invention, the subject is administered a further chemotherapeutic agent (e.g., an anti-cancer chemotherapeutic agent) or an anti-cancer therapeutic procedure. In an embodiment of the invention, the anti-cancer therapeutic procedure is anti-cancer radiation therapy or surgical tumorectomy. In an embodiment of the invention, the further chemotherapeutic agent is one or more members selected from the group consisting of: everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a BcI-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380,

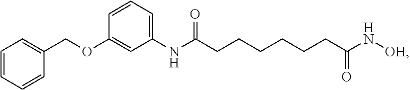

### CG-781, CG-1521,

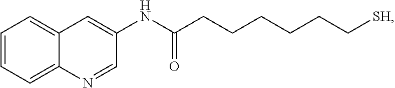

SB-556629, chlamydocin, JNJ-16241199,

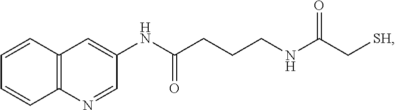

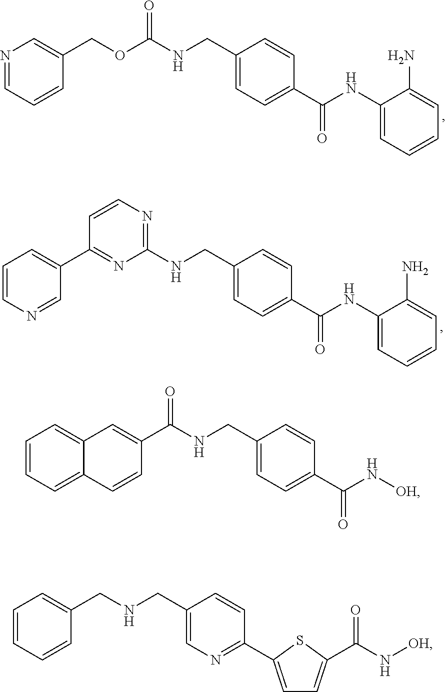

vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258,

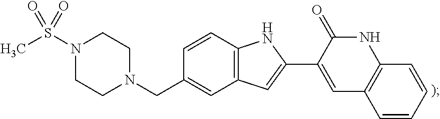

3-[5-(methylsulfonylpiperadinemethyl)-indolyl]-quinolone, vatalanib, AG-013736, AVE-0005, the acetate salt of [D-Ser(Bu t)6,Azgly 10](pyro-Glu-His-Trp-Ser-Tyr-D-Ser(Bu t)-Leu-Arg-Pro-Azgly-NH₂ acetate[C₅₉H₈₄N₁₈O₁₄—(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib,

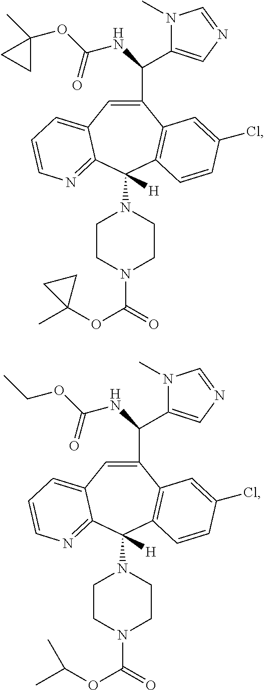

BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3 (metastat), neovastat, BMS-275291

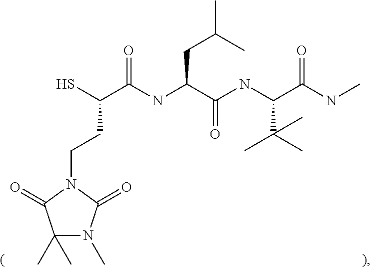

squalamine, endostatin, SU5416 (semaxinib), SU6668 ([(Z)-3-[2,4-dimethyl-5-(2-oxo-1,2-dihydro-indol-3-ylidenemethyl)-1H-pyrrol-3-yl]-propionic acid), EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550 (ixabepilone), BMS-310705

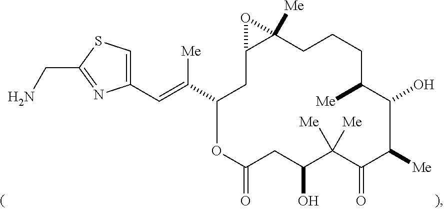

droloxifene, 4-hydroxytamoxifen, pipendoxifene, ERA-923 (2-(4-Hydroxy-phenyl)-3-methyl-1-[4-(2-piperidin-1-yl-ethoxy)-benzyl]-1H-indol-5-ol hydrochloride), arzoxifene, fulvestrant, acolbifene, lasofoxifene, idoxifene, TSE-424 (bazedoxifene acetate), HMR-3339 (4-chloro-11b-[4-(2-[diethylamino]ethoxy)phenyl]-estra-1,3,5(10)-triene-3,17b-diol), ZK186619, topotecan, PTK787/ZK 222584, VX-745

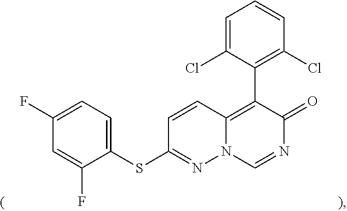

PD184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa. In an embodiment of the invention, the antibody or fragment, the leucovorin, the 5-fluorouracil and the sunitinib are in separate pharmaceutical compositions, each independently further comprising a pharmaceutically acceptable carrier. In an embodiment of the invention, the subject suffers from one or more conditions selected from the group consisting of: familial adenomatous polyposis, hereditary nonpolyposis colon cancer, Lynch I Syndrome, Lynch II Syndrome, inflammatory bowel disease, chronic ulcerative colitis (UC), Crohn's disease, a family cancer syndrome, Peutz-Jegher Syndrome, familial juvenile polyposis and one or more adenomatous polyps.

The present invention further includes within its scope a combination or composition comprising an isolated antibody or antigen-binding fragment thereof comprising one or more members selected from the group consisting of: (a) CDR-L1, CDR-L2 and CDR-L3 of the variable region of light chain C, light chain D, light chain E or light chain F; or (b) CDR-H1, CDR-H2 and CDR-H3 of the variable region of heavy chain A or heavy chain B; or both; in association with (i) leucovorin and 5-fluorouracil; (ii) leucovorin; or (iii) sunitinib; optionally in further association with a further chemotherapeutic agent (e.g., a further chemotherapeutic agent set forth herein). For example, in an embodiment of the invention, the antibody or fragment comprises a light chain variable region comprising amino acids 20-128 of SEQ ID NO: 9, 11, 13 or 15 and/or a heavy chain variable region comprising amino acids 20-137 of SEQ ID NO: 17 or 19.

### DETAILED DESCRIPTION OF THE INVENTION

The present invention provides, inter alia, methods for treating or preventing colorectal cancer in a subject in need of such treatment or prevention, by administering a 15H12/19D12 anti-IGF1R antibody or antigen-binding fragment thereof of the invention in association with leucovorin and 5-fluorouracil or in association with sunitinib. In an embodiment of the invention, no other components are administered to a subject as part of a method or therapeutic treatment regimen of the present invention. In an embodiment of the invention, one or more further chemotherapeutic agents are administered to the subject.

### Antibodies and Antigen-Binding Fragments Thereof

In an embodiment of the invention, subjects are administered any antibody or antigen-binding fragment thereof, e.g., that specifically binds to IGF1R, which comprises light chain CDRs or heavy chain CDRs or both, for example, as set forth below:

15H12/19D12 light chain immunoglobulin CDRs

| | |
|---|---|
| CDR-L1: RASQSIGSSLH | (SEQ ID NO: 1) |
| CDR-L2: YASQSLS; | (SEQ ID NO: 2) |
| CDR-L3: HQSSRLPHT; | (SEQ ID NO: 3) |

for example, all three light chain immunoglobulin CDRs;
and/or
15H12/19D12 heavy chain immunoglobulin CDRs

| | | |
|---|---|---|
| CDR-H1: | SFAMH; | (SEQ ID NO: 4) |
| or | | |
| | GFTFSSFAMH; | (SEQ ID NO: 5) |
| CDR-H2: | VIDTRGATYYADSVKG; | (SEQ ID NO: 6) |
| CDR-H3: | LGNFYYGMDV; | (SEQ ID NO: 7) |

for example, all three heavy chain immunoglobulin CDRs.

In an embodiment of the invention, the antibody comprises any combination of the following light and heavy chain immunoglobulin chains (e.g., mature fragments thereof). Signal sequences are underscored with dashed lines and CDR sequences are underscored by solid lines. In an embodiment of the invention, mature variable region fragments lack the signal sequences.

15H12/19D12 immunoglobulin light chain-C (LCC)

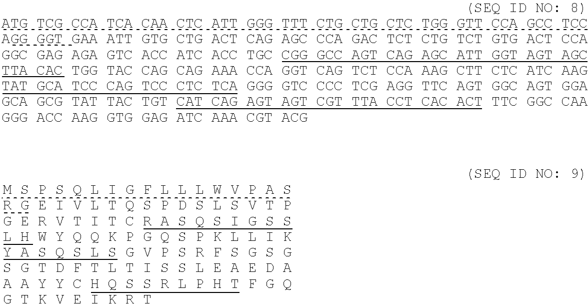

15H12/19D12 immunoglobulin light chain-D (LCD)

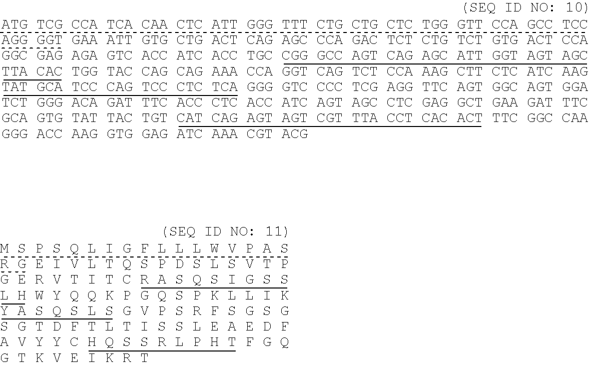

15H12/19D12 immunoglobulin light chain-E (LCE)

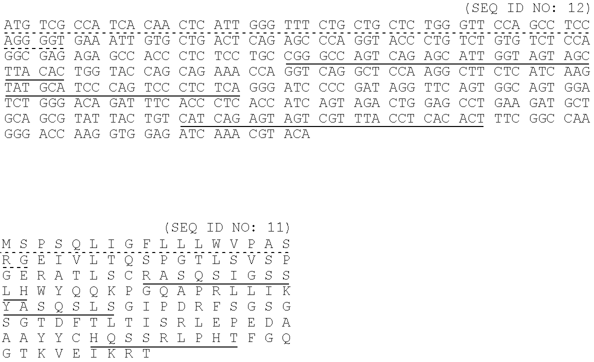

15H12/19D12 immunoglobulin light chain-F (LCF)

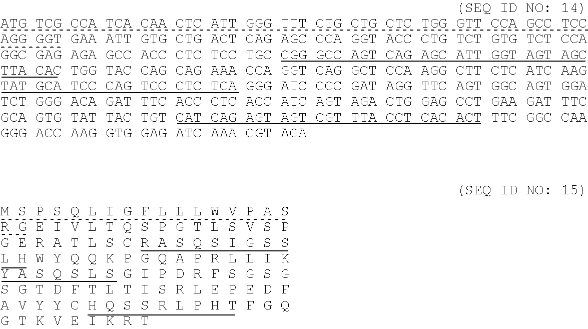

15H12/19D12 immunoglobulin heavy chain-A (HCA)

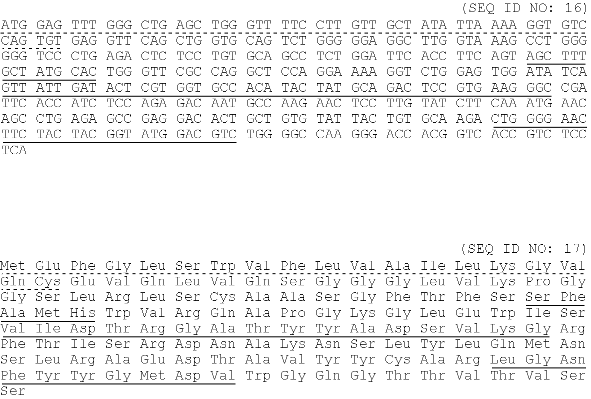

15H12/19D12 immunoglobulin heavy chain-B (HCB)

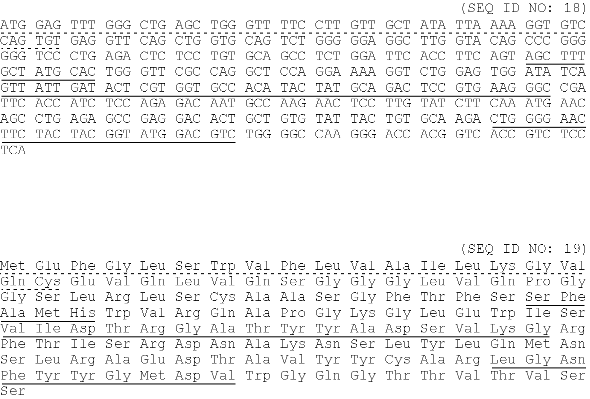

See U.S. Pat. No. 7,217,796; any anti-IGF1R or antigen-binding fragment thereof in the patent can be used in a method of the present invention.

In an embodiment of the present invention, the anti-IGF1R antibody light chain and/or heavy chain is encoded by any plasmid selected from the group consisting of:

(i) CMV promoter-15H12/19D12 HCA (γ4)— Deposit name: “15H12/19D12 HCA (γ4)”; ATCC accession No.: PTA-5214;
(ii) CMV promoter-15H12/19D12 HCB γ4)— Deposit name: “15H12/19D12 HCB (γ4)”; ATCC accession No.: PTA-52 15;
(iii) CMV promoter-15H12/19D12 HCA (γ1)— Deposit name: “15H12/19D12 HCA (γ1)”; ATCC accession No.: PTA-5216;
(iv) CMV promoter-15H12/19D12 LCC (κ)— Deposit name: “15H12/19D12 LCC (κ)”; ATCC accession No.: PTA-5217;
(v) CMV promoter-15H12/19D12 LCD (κ)— Deposit name: “15H12/19D12 LCD (κ)”; ATCC accession No.: PTA-5218;
(vi) CMV promoter-15H12/19D12 LCE (κ)— Deposit name: “15H12/19D12 LCE (κ)”; ATCC accession No.: PTA-5219; and
(vii) CMV promoter-15H12/19D12 LCF (κ)— Deposit name: “15H12/19D12 LCF (κ)”, ATCC accession No.: PTA-5220;

The above-identified plasmids were deposited, under the Budapest Treaty, on May 21, 2003, with the American Type Culture Collection (ATCC); 10801 University Boulevard; Manassas, Va. 20110 2209. All restrictions on the accessibility of the deposited plasmids to the public have been irrevocably removed by the applicant.

In an embodiment of the invention, the antibody is an LCC/HCA, LCD/HCB or LCF/HCA.

In an embodiment of the invention, the anti-IGF1R antibody or antigen-binding fragment thereof comprises the mature heavy chain immunoglobulin variable region:

(SEQ ID NO: 20)
vqllesggglvqpggslrlsctasgftfssyamnwvrqapgkglewvs
aisgsggttfyadsvkgrftisrdnsrttylqmnslraedtavyycak
dlgwsdsyyyyygmdvwgqgttvtvss;

or one or more CDRs (e.g., 3) therefrom.

In an embodiment of the invention, the anti-IGF1R antibody or antigen-binding fragment thereof comprises the mature light chain immunoglobulin variable region:

(SEQ ID NO: 21)
diqmtqfpsslsasvgdrytitcrasqgirndlgwyqqkpgkapkrli
yaasrlhrgypsrfsgsgsgteftltisslqpedfatyyclqhnsypc
sfgqgtkleik;

or one or more CDRs (e.g., 3) therefrom.

The present invention includes methods for using anti-IGF1R antibodies and antigen-binding fragments thereof. Thus, the invention includes methods for using monoclonal antibodies, camelized single domain antibodies, polyclonal antibodies, bispecific antibodies, chimeric antibodies, recombinant antibodies, anti-idiotypic antibodies, humanized antibodies, bispecific antibodies, diabodies, single chain antibodies, disulfide Fvs (dsfv), Fvs, Fabs, Fab′ s, F(ab′)₂s and domain antibodies. Thus, the term “antibody” and the like covers, but is not limited to, monoclonal antibodies, polyclonal antibodies, recombinant antibodies, multispecific antibodies (e.g., bispecific antibodies). The term “antigen-binding fragment” and the like of an antibody (of the “parental antibody”) encompasses a fragment or a derivative of an antibody, typically including at least a portion of the antigen-binding or variable region (e.g., one or more CDRs) of the parental antibody, that retains at least some of the binding specificity of the parental antibody. Examples of antibody antigen-binding fragments include, but are not limited to, Fab, Fab′, F(ab′)₂, and Fv fragments; diabodies; single-chain antibody molecules, e.g., sc-Fv; and multispecific antibodies formed from antibody fragments. Typically, a binding fragment or derivative retains at least 10% of its IGF1R binding activity when that activity is expressed on a molar basis. In an embodiment of the invention, a binding fragment or derivative retains at least 20%, 50%, 70%, 80%, 90%, 95% or 100% or more of the IGF1R binding affinity as the parental antibody. It is also intended that an antigen-binding fragment can include conservative amino acid substitutions (referred to as “conservative variants” of the antibody) that do not substantially alter its biologic activity.

In an embodiment of the invention, “Fab” refers to a fragment including a single light chain (both variable and constant regions) bound to the variable region and first constant region of a single heavy chain by a disulfide bond. Fab fragments may be produced by, for example, papain digestion of an IgG antibody.

In an embodiment of the invention, “Fab′” refers to a Fab fragment that includes a portion of the hinge region.

In an embodiment of the invention, “F(ab′)₂” refers to a dimer of Fab′. F(ab′)₂ fragments which may be produced by enzymatic cleavage of an IgG by, for example, pepsin. A Fab′ may be generated, for example, by reduction of a F(ab′)₂ with, e.g., 2-mercaptoethanol.

In an embodiment of the invention, “Fc” refers to that portion of the antibody consisting of the second and third constant regions of a first heavy chain bound to the second and third constant regions of a second heavy chain via disulfide bonding. The Fc portion of the antibody is responsible for various effector functions such as ADCC, and CDC, but does not function in antigen binding.

In an embodiment of the invention, “Fv”, with regards to an antibody, is the variable region of a single light chain bound to the variable region of a single heavy chain.

In an embodiment of the invention, a “disulfide stabilized Fv fragment” or “dsFv” comprises molecules having a variable heavy chain (V_{H}) and/or a variable light chain (V_{L}) which are linked by a disulfide bridge.

In an embodiment of the invention, the term “single-chain Fv” or “scFv” antibody comprises antibody fragments comprising the V_{H} and V_{L} domains of an antibody, wherein these domains are present in a single polypeptide chain. Generally, the Fv polypeptide further comprises a polypeptide linker between the V_{H} and V_{L} domains which enables the V_{H} and V_{L} chains to pair and form a binding site (e.g., 5-12 residues long). For a review of scFv, see Pluckthun (1994) THE PHARMACOLOGY OF MONOCLONAL ANTIBODIES, vol. 113, Rosenburg and Moore eds. Springer-Verlag, New York, pp. 269-315. See also, International Patent Application Publication No. WO 88/01649 and U.S. Pat. Nos. 4,946,778 and 5,260,203.

In an embodiment of the invention, a “single-chain Fv-Fc antibody” or “scFv-Fc” refers to an engineered antibody including a scFv connected to the Fc region of an antibody.

In an embodiment of the invention, a “nanobody” the VHH domain of a heavy-chain antibodies. Such heavy chain antibodies contain a single variable domain (VHH) and two constant domains (CH2 and CH3).

In an embodiment of the invention, a “domain antibody” (e.g., V_{L} domain or V_{H} domain) comprises an immunologically functional immunoglobulin fragment containing only the variable region of a heavy chain or the variable region of a light chain. In some instances, two or more V_{H} regions are covalently joined with a peptide linker to create a bivalent domain antibody. The two V_{H} regions of a bivalent domain antibody may target the same or different antigens.

In an embodiment of the invention, a “bivalent” or “bispecific” antibody comprises two antigen-binding sites. In some instances, the two binding sites have the same antigen specificities. However, bivalent antibodies may be bispecific. For example, the present invention comprises full antibodies, scfv dimers and dsfv dimers having a single or different antigen binding specificities.

In an embodiment of the invention, a (dsfv)₂ comprises three peptide chains: two V_{H} moieties linked by a peptide linker and bound by disulfide bridges to two V_{L} moieties. In an embodiment of the invention, a bispecific ds diabody comprises a VH₁-VL₂ (tethered by a peptide linker) linked, by a disulfide bridge between the VH₁ and VL₁, to a VL₁-VH₂ moiety (also tethered by a peptide linker). In an embodiment of the invention, a bispecific dsfv-dsfv′ also comprises three peptide chains: a VH₁-VH₂ moiety wherein the heavy chains are linked by a peptide linker (e.g., a long flexible linker) and are bound to VL₁ and VL₂ moieties, respectively, by disulfide bridges; wherein each disulfide paired heavy and light chain has a different antigen specificity. In an embodiment of the invention, an scfv dimer (a bivalent diabody) comprises a V_{H}-V_{L} moiety wherein the heavy and light chains are bound to by a peptide linker and dimerized with another such moiety such that V_{H}s of one chain coordinate with the V_{L}s of another chain and form two identical binding sites. In an embodiment of the invention a bispecific diabody comprises VH₁-VL₂ moiety (linked by a peptide linker) associated with a VL₁-VH₂ (linked by a peptide linker), wherein the VH₁ and VL₁ coordinate and the VH₂ and VL₂ coordinate and each coordinated set has diverse antigen specificities.

In an embodiment of the invention, the term “monoclonal antibody” comprises an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. In contrast, conventional (polyclonal) antibody preparations typically include a multitude of antibodies directed against (or specific for) different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made recombinantly or by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al., (1991) Nature 352: 624-628 and Marks et al. (1991) J. Mol. Biol. 222: 581-597, for example. See also Presta (2005) J. Allergy Clin. Immunol. 116:731.

Monoclonal antibodies include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., (1984) Proc. Natl. Acad. Sci. USA 81: 6851-6855). For example, variable domains are obtained from an antibody from an experimental animal (the “parental antibody”), such as a human, and the constant domain sequences are obtained from canine antibodies, so that the resulting chimeric antibody will be less likely to elicit an adverse immune response in a canine subject than the parental human antibody.

In an embodiment of the invention, a recombinant antibody or antigen-binding fragment thereof of the invention is an antibody which is produced recombinantly, e.g., expressed from a polynucleotide which has been introduced into an organism (e.g., a plasmid containing a polynucleotide encoding the antibody or fragment transformed into a bacterial cell (e.g., E. coli) or a mammalian cell (e.g., CHO cell)), followed by isolation of the antibody or fragment from the organism.

In an embodiment of the invention, anti-idiotypic antibodies or anti-idiotypes are antibodies directed against the antigen-combining region or variable region (called the idiotype) of another antibody molecule. As disclosed by Jerne (Jerne, N. K., (1974) Ann. Immunol. (Paris) 125c:373 and Jerne, N. K., et al., (1982) EMBO 1:234), immunization with an antibody molecule expressing a paratope (antigen-combining site) for a given antigen will produce a group of anti-antibodies, some of which share, with the antigen, a complementary structure to the paratope. Immunization with a subpopulation of the anti-idiotypic antibodies will, in turn, produce a subpopulation of antibodies or immune cell subsets that are reactive to the initial antigen.

The present invention also includes camelized single domain antibodies. See, e.g., Muyldermans et al. (2001) Trends Biochem. Sci. 26:230; Reichmann et al. (1999) J. Immunol. Methods 231:25; WO 94/04678; WO 94/25591; U.S. Pat. No. 6,005,079, which are hereby incorporated by reference in their entireties). Camelidae (camels, dromedaries and llamas) comprise IgG antibodies in which are devoid of light chains and therefore called ‘heavy-chain’ IgGs or HCAb (for heavy-chain antibody). HCAbs typically have a molecular weight of ˜95 kDa since they consist only of the heavy-chain variable domains. Although the HCAbs are devoid of light chains, they have an authentic antigen-binding repertoire (Hamers-Casterman et al., Nature (1993) 363:446-448; Nguyen et al., Adv. Immunol. (2001) 79:261-296; Nguyen et al., Immunogenetics. (2002) 54:39-47). In one embodiment, the present invention provides single domain antibodies comprising two V_{H} domains with modifications such that single domain antibodies are formed.

In an embodiment of the invention, the term “diabodies” includes small antibody fragments with two antigen-binding sites, which fragments comprise a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., EP 404,097; WO 93/11161; and Holliger et al. (1993)Proc. Natl. Acad. Sci. USA 90: 6444-6448. For a review of engineered antibody variants generally see Holliger and Hudson (2005) Nat. Biotechnol. 23:1126-1136.

In an embodiment of the invention, the term “humanized antibody” comprises forms of antibodies that contain sequences from both human and non-human (e.g., murine, rat) antibodies. In general, the humanized antibody will comprise substantially all of at least one, and typically two, variable domains, in which all or substantially all of the hypervariable loops correspond to those of a non-human immunoglobulin, and all or substantially all of the framework (FR) regions are those of a human immunoglobulin sequence. The humanized antibody may optionally comprise at least a portion of a human immunoglobulin constant region (Fc).

For example, the present invention comprises any humanized antibody comprising the CDRs of 15H12/19D12, e.g., wherein identical CDRs were originally isolated from a non-human species antibody and incorporated into a human antibody framework.

The antibodies of the present invention also include antibodies with modified (or blocked) Fc regions to provide altered effector functions. See, e.g., U.S. Pat. No. 5,624,821; WO2003/086310; WO2005/120571; WO2006/0057702. Such modifications can be used to enhance or suppress various reactions of the immune system, with possible beneficial effects in diagnosis and therapy. Alterations of the Fc region include amino acid changes (substitutions, deletions and insertions), glycosylation or deglycosylation, and adding multiple Fc. Changes to the Fc can also alter the half-life of antibodies in therapeutic antibodies, enabling less frequent dosing and thus increased convenience and decreased use of material. See Presta (2005)J. Allergy Clin. Immunol. 116:731 at 734-35.

The anti-IGF1R antibodies and antigen-binding fragments thereof of the invention are, in an embodiment of the invention, conjugated to a chemical moiety. The chemical moiety may be, inter alia, a polymer, a radionuclide or a cytotoxic factor. In an embodiment of the invention, the chemical moiety is a polymer which increases the half-life of the antibody or fragment in the body of a subject to whom it is administered. Polymers include, but are not limited to, polyethylene glycol (PEG) (e.g., PEG with a molecular weight of 2 kDa, 5 kDa, 10 kDa, 12 kDa, 20 kDa, 30 kDa or 40 kDa), dextran and monomethoxypolyethylene glycol (mPEG). Lee, et al., (1999) (Bioconj. Chem. 10:973-981) discloses PEG conjugated single-chain antibodies. Wen, et al., (2001) (Bioconj. Chem. 12:545-553) disclose conjugating antibodies with PEG which is attached to a radiometal chelator (diethylenetriaminepentaacetic acid (DTPA)).

The antibodies and antigen-binding fragments of the invention are, in an embodiment of the invention, conjugated with labels such as ^{99m}Tc, ⁹⁹Tc, ⁹⁰Y, ¹¹¹In, ³²P, ¹⁴C, ¹²⁵L, ³H, ¹³¹I, ¹²³I, ¹¹C, ¹⁵O, ¹³N, ¹⁸F, ³⁵S, ⁵¹Cr, ⁵⁷To, ²²⁶Ra, ⁶⁰Co, ⁵⁹Fe, ⁵⁷ Se, ¹⁵²Eu, ⁶⁷Cu, ²¹⁷Ci, ²¹¹At, ²¹²Pb, ⁴⁷Sc, ¹⁰⁹Pd, ²³⁴Th, and ⁴⁰K, ¹⁵⁷Gd, ⁵⁵Gd, ⁵⁵Mn, ⁵²Tr and ⁵⁶Fe.

The antibodies and antigen-binding fragments of the invention may also be conjugated with fluorescent or chemiluminescent labels, including fluorophores such as rare earth chelates, fluorescein and its derivatives, rhodamine and its derivatives, isothiocyanate, phycoerythrin, phycocyanin, allophycocyanin, o-phthaladehyde, fluorescamine, ¹⁵²Eu, dansyl, umbelliferone, luciferin, luminal label, isoluminal label, an aromatic acridinium ester label, an imidazole label, an acridimium salt label, an oxalate ester label, an aequorin label, 2,3-dihydrophthalazinediones, biotin, avidin, peroxidase such as horseradish peroxidase, alkaline phosphatase (e.g., calf, shrimp or bacterial), spin labels and stable free radicals.

The antibodies and antigen-binding fragments of the invention may also be conjugated to a cytotoxic factor such as diptheria toxin, Pseudomonas aeruginosa exotoxin A chain, ricin A chain, abrin A chain, modeccin A chain, alpha-sarcin, Aleurites fordii proteins and compounds (e.g., fatty acids), dianthin proteins, Phytoiacca americana proteins PAPI, PAPII, and PAP-S, momordica charantia inhibitor, curcin, crotin, saponaria officinalis inhibitor, mitogellin, restrictocin, phenomycin, and enomycin.

Any method known in the art for conjugating the antibodies and antigen-binding fragments of the invention to the various moieties may be employed, including those methods described by Hunter, et al., (1962) Nature 144:945; David, et al., (1974) Biochemistry 13:1014; Pain, et al., (1981) J. Immunol. Meth. 40:219; and Nygren, J., (1982) Histochem. and Cytochem. 30:407. Methods for conjugating antibodies are conventional and very well known in the art.

### Generation of Antibodies

Any suitable method for generating antibodies or antigen-binding fragments thereof, e.g., monoclonal antibodies, may be used. The present invention includes both recombinant and non-recombinant methods of production, e.g., as discussed herein. Non-recombinant methods include immunization of animals and subsequent isolation of antibodies or splenocytes (e.g., followed by hybridoma production) from the immunized animal. For example, a recipient may be immunized with a linked or unlinked (e.g., naturally occurring) form of IGF1R, or a fragment thereof. Any suitable method of immunization can be used. Such methods can include adjuvants, other immunostimulants, repeated booster immunizations, and the use of one or more immunization routes.

In an embodiment of the invention, human monoclonal antibodies directed against IGF1R are generated using transgenic mice carrying parts of the human immune system rather than the mouse system. These transgenic mice, which may be referred to, herein, as “HuMAb” mice, contain human immunoglobulin gene miniloci that encodes unrearranged human heavy (μ and γ) and κ light chain immunoglobulin sequences, together with targeted mutations that inactivate the endogenous μ and κ chain loci (Lonberg, N., et al., (1994) Nature 368(6474): 856-859). Accordingly, the mice exhibit reduced expression of mouse IgM or κ, and in response to immunization, the introduced human heavy and light chain transgenes undergo class switching and somatic mutation to generate high affinity human IgGκ monoclonal antibodies (Lonberg, N., et al., (1994), supra; reviewed in Lonberg, N. (1994) Handbook of Experimental Pharmacology 113:49-101; Lonberg, N., et al., (1995) Intern. Rev. Immunol. 13:65-93, and Harding, F., et al., (1995) Ann. N.Y. Acad. Sci. 764:536-546). The preparation of HuMab mice is commonly known in the art and is described, for example, in Taylor, L., et al., (1992) Nucleic Acids Research 20:6287-6295; Chen, J., et al., (1993) International Immunology 5: 647-656; Tuaillon, et al., (1993) Proc. Natl. Acad. Sci. USA 90:3720-3724; Choi, et al., (1993) Nature Genetics 4:117-123; Chen, J., et al., (1993) EMBO J. 12: 821-830; Tuaillon, et al., (1994) J. Immunol. 152:2912-2920; Lonberg, et al., (1994) Nature 368(6474): 856-859; Lonberg, N. (1994) Handbook of Experimental Pharmacology 113:49-101; Taylor, L., et al., (1994) International Immunology 6: 579-591; Lonberg, N., et al., (1995) Intern. Rev. Immunol. Vol. 13: 65-93; Harding, F., et al., (1995) Ann. N.Y. Acad. Sci. 764:536-546; Fishwild, D., et al., (1996) Nature Biotechnology 14: 845-851 and Harding, et al., (1995) Annals NY Acad. Sci. 764:536-546; the contents of all of which are hereby incorporated by reference in their entirety. See further, U.S. Pat. Nos. 5,545,806; 5,569,825; 5,625,126; 5,633,425; 5,789,650; 5,877,397; 5,661,016; 5,814,318; 5,874,299; 5,770,429 and 5,545,807; and International Patent Application Publication Nos. WO 98/24884; WO 94/25585; WO 93/12227; WO 92/22645 and WO 92/03918 the disclosures of all of which are hereby incorporated by reference in their entity. The use of HuMAb mice is commercially available from Medarex, Inc. (Princeton, N.J.).

To generate fully human, monoclonal antibodies to IGF1R, HuMab mice can be immunized with an antigenic IGF1R polypeptide, as described by Lonberg, N., et al., (1994) Nature 368(6474): 856-859; Fishwild, D., et al., (1996) Nature Biotechnology 14: 845-851 and WO 98/24884. In an embodiment of the invention, the mice will be 6-16 weeks of age upon the first immunization. For example, a purified preparation of IGF1R or sIGF1R can be used to immunize the HuMab mice intraperitoneally. The mice can also be immunized with whole HEK293 cells which are stably transformed or transfected with an IGF1R gene.

In general, HuMAb transgenic mice respond well when initially immunized intraperitoneally (i.p.) with antigen in complete Freund's adjuvant, followed by every other week IP immunizations (usually, up to a total of 6) with antigen in incomplete Freund's adjuvant. Mice can be immunized, first, with cells expressing IGF1R (e.g., stably transformed HEK293 cells), then with a soluble fragment of IGF1R and continually receive alternating immunizations with the two antigens. The immune response can be monitored over the course of the immunization protocol with plasma samples being obtained by retroorbital bleeds. The plasma can be screened for the presence of anti-IGF1R antibodies, for example by ELISA, and mice with sufficient titers of immunoglobulin can be used for fusions. Mice can be boosted intravenously with antigen 3 days before sacrifice and removal of the spleen. Several mice can be immunized for each antigen.

Hybridoma cells which produce the monoclonal, fully human anti-IGF1R antibodies may be produced by methods which are commonly known in the art. These methods include, but are not limited to, the hybridoma technique originally developed by Kohler, et al., (1975) (Nature 256:495-497), as well as the trioma technique (Hering, et al., (1988) Biomed. Biochim. Acta. 47:211-216 and Hagiwara, et al., (1993) Hum. Antibod. Hybridomas 4:15), the human B-cell hybridoma technique (Kozbor, et al., (1983) Immunology Today 4:72 and Cote, et al., (1983) Proc. Natl. Acad. Sci. U.S.A 80:2026-2030), and the EBV-hybridoma technique (Cole, et al., in Monoclonal Antibodies and Cancer Therapy, Alan R. Liss, Inc., pp. 77-96, 1985). In an embodiment of the invention, mouse splenocytes are isolated and fused with PEG to a mouse myeloma cell line based upon standard protocols. The resulting hybridomas are then screened for the production of antigen-specific antibodies. For example, single cell suspensions of splenic lymphocytes from immunized mice may by fused to one-sixth the number of P3X63-Ag8.653 nonsecreting mouse myeloma cells (ATCC, CRL 1580) with 50% PEG. Cells are plated at approximately 2×10⁵ cells/mL in a flat bottom microtiter plate, followed by a two week incubation in selective medium containing 20% fetal Clone Serum, 18% “653” conditioned media, 5% origen (IGEN), 4 mM L-glutamine, 1 mM L-glutamine, 1 mM sodium pyruvate, 5 mM HEPES, 0.055 mM 2-mercaptoethanol, 50 units/ml penicillin, 50 mg/ml streptomycin, 50 mg/ml gentamycin and 1×HAT (Sigma; the HAT is added 24 hours after the fusion). After two weeks, cells are cultured in medium in which the HAT is replaced with HT. Individual wells are then screened by ELISA for identification of human anti-IGF1R monoclonal IgG antibodies. Once extensive hybridoma growth occurs, medium can be observed usually after 10-14 days. The antibody secreting hybridomas may be replated, screened again, and if still positive for human IgG, anti-IGF1R monoclonal antibodies, can be subcloned at least twice by limiting dilution. The stable subclones may then be cultured in vitro to generate small amounts of antibody in tissue culture medium for characterization.

In general, for recombinant production of an immunoglobulin chain, a nucleic acid encoding it is isolated and inserted into a replicable vector for further cloning (amplification of the DNA) and/or for expression. DNA encoding the chain is readily isolated and sequenced using conventional procedures (e.g., by using oligonucleotide probes that are capable of binding specifically to genes encoding the heavy and light chains of the antibody). Many vectors are available. The vector components generally include, but are not limited to, one or more of the following: a signal sequence, an origin of replication, one or more marker genes, an enhancer element, a promoter, and a transcription termination sequence.

Recombinant immunoglobulins may be produced, e.g., by the method of Cabilly U.S. Pat. No. 4,816,567; and Queen et al. (1989) Proc. Nat'l Acad. Sci. USA 86:10029-10033; or made in transgenic mice, see Mendez et al. (1997) Nature Genetics 15:146-156. A recombinant method may comprise preparing a DNA sequence encoding an immunoglobulin heavy or light chain having specificity for a particular antigen; inserting the sequence into a replicable expression vector operably linked to a suitable promoter compatible with a host cell (e.g., bacterial cell such as E. coli or a mammalian cell); transforming the host cell with the vector; culturing the host cell; and recovering the heavy or light chain from the host cell culture.

In one embodiment of the invention, the antibodies or fragments of the present invention are produced in yeast according to the methods described in published international patent application no. WO2005/040395. Briefly, vectors encoding the individual light or heavy chains of an antibody of interest are introduced into different yeast haploid cells, e.g. different mating types of the yeast Pichia pastoris, which yeast haploid cells are optionally complementary auxotrophs. The transformed haploid yeast cells can then be mated or fused to give a diploid yeast cell capable of producing both the heavy and the light chains. The diploid strain is then able to secrete the fully assembled and biologically active antibody. The relative expression levels of the two chains can be optimized, for example, by using vectors with different copy numbers, using transcriptional promoters of different strengths, or inducing expression from inducible promoters driving transcription of the genes encoding one or both chains.

In an embodiment of the present invention, the respective heavy and light chains of a plurality of different anti-IGF1R antibodies (the “original” antibodies) are introduced into yeast haploid cells to create a library of haploid yeast strains of one mating type expressing a plurality of light chains, and a library of haploid yeast strains of a different mating type expressing a plurality of heavy chains. These libraries of haploid strains can be mated (or fused as spheroplasts) to produce a series of dipoid yeast cells expressing a combinatorial library of antibodies comprised of the various possible permutations of light and heavy chains. The combinatorial library of antibodies can then be screened to determine whether any of the antibodies has properties that are superior (e.g., higher affinity for IGF1R) to those of the original antibodies.

In an embodiment of the invention, immunoglobulin chains are generated by any of the recombinant immunoglobulin production methods set forth in published U.S. patent application no. US2005/0176099 to D. Saha (see also U.S. Pat. No. 7,326,567).

### Further Chemotherapeutic Agents

The present invention comprises methods where a subject is administered an anti-IGF1R antibody or antigen-binding fragment thereof in association with leucovorin and 5-fluorouracil or in association with sunitinib. Moreover, the present invention further comprises combinations including such antibodies or fragments in association with (i) leucovorin, (ii) sunitinib or (iii) leucovorin and 5-fluorouracil; optionally in further association with a further chemotherapeutic agent. Further therapeutic agents include, e.g., one or more anti-cancer therapeutic agents or one or more of: a FLT-3 inhibitor, a VEGFR inhibitor, an EGFR TK inhibitor, an aurora kinase inhibitor, an mTOR inhibitor, a PIK-1 modulator, a BcI-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor or a VEGF trap antibody.

In an embodiment of the invention, the further chemotherapeutic agent is one or more of: everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380,

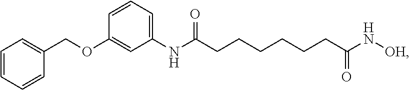

### CG-781, CG-1521,

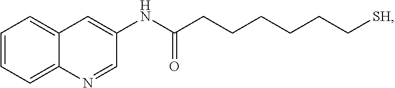

SB-556629, chlamydocin, JNJ-16241199,

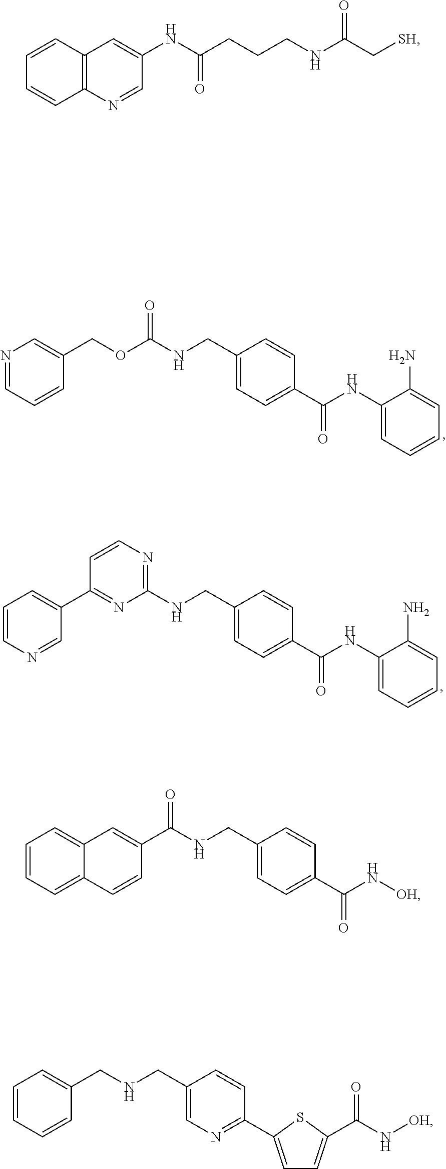

vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258,

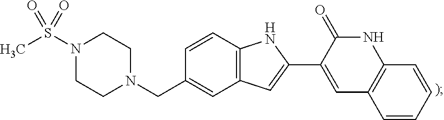

3-[5-(methylsulfonylpiperadinemethyl)-indolyl]-quinolone, vatalanib, AG-013736, AVE-0005, the acetate salt of [D-Ser(Bu t)6,Azgly 10](pyro-Glu-His-Trp-Ser-Tyr-D-Ser(Bu t)-Leu-Arg-Pro-Azgly-NH₂ acetate[C₅₉H₈₄N₁₈O₁₄—(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib,

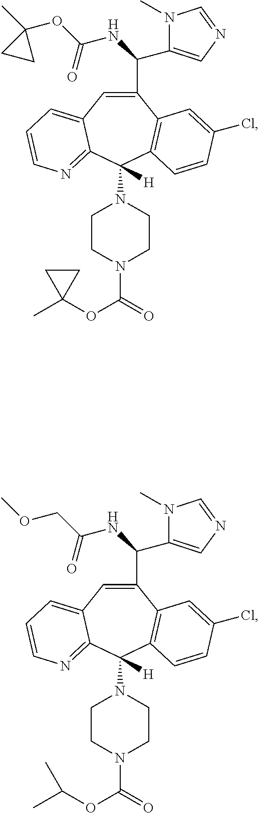

BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel or docetaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

Nausea is an unpleasant side effect of many anti-cancer treatments. Accordingly, in part to address this issue, the scope of the present invention also includes methods wherein a subject is administered an anti-IGF1R antibody or antigen-binding fragment thereof in association with leucovorin and 5-fluorouracil or in association with sunitinib; further in association with one or more antiemetics. Antiemetics include, but are not limited to, casopitant (GlaxoSmithKline), Netupitant (MGI-Helsinn) and other NK-1 receptor antagonists, palonosetron (sold as Aloxi by MGI Pharma), aprepitant (sold as Emend by Merck and Co.; Rahway, N.J.), diphenhydramine (sold as Benadryl® by Pfizer; New York, N.Y.), hydroxyzine (sold as Atarax® by Pfizer; New York, N.Y.), metoclopramide (sold as Reglan® by AH Robins Co; Richmond, Va.), lorazepam (sold as Ativan® by Wyeth; Madison, N.J.), alprazolam (sold as Xanax® by Pfizer; New York, N.Y.), haloperidol (sold as Haldol® by Ortho-McNeil; Raritan, N.J.), droperidol (Inapsine®), dronabinol (sold as Marinol® by Solvay Pharmaceuticals, Inc.; Marietta, Ga.), dexamethasone (sold as Decadron® by Merck and Co.; Rahway, N.J.), methylprednisolone (sold as Medrol® by Pfizer; New York, N.Y.), prochlorperazine (sold as Compazine® by Glaxosmithkline; Research Triangle Park, N.C.), granisetron (sold as Kytril® by Hoffmann-La Roche Inc.; Nutley, N.J.), ondansetron (sold as Zofran® by Glaxosmithkline; Research Triangle Park, N.C.), dolasetron (sold as Anzemet® by Sanofi-Aventis; New York, N.Y.), tropisetron (sold as Navoban® by Novartis; East Hanover, N.J.).

Other side effects of cancer treatment include red and white blood cell deficiency. Accordingly, the present invention includes methods wherein the subject is administered an anti-IGF1R antibody or antigen-binding fragment thereof in association with leucovorin and 5-fluorouracil or in association with sunitinib; further in association with an agent which treats or prevents such a deficiency, such as, e.g., pegfilgrastim, erythropoietin, epoetin alfa or darbepoetin alfa.

The term “in association with” indicates that the components of a composition which are administered in connection with a method of the present invention can be formulated into a single composition for simultaneous delivery or formulated separately into two or more compositions (e.g., a kit). Furthermore, each component can be administered to a subject at a different time than when the other component is administered; for example, each administration may be given non-simultaneously (e.g., separately or sequentially) at one or more intervals over a given period of time. Moreover, the separate components may be administered to a subject by the same or by a different route.

As discussed above, in an embodiment of the invention, a subject is administered an anti-IGF1R antibody or antigen-binding fragment thereof of the invention in association with leucovorin and 5-fluorouracil or in association with sunitinib and, optionally in association with a further chemotherapeutic agent, such as a further anti-IGF1R antibody or antigen-binding fragment thereof. In an embodiment of the invention, however, said further chemotherapeutic agent is another anti-IGF1R antibody or antigen binding fragment thereof with the proviso that the antibody or fragment does not comprise a 2C6 light chain or heavy chain immunoglobulin or a 9H2 light chain or heavy chain immunoglobulin or any CDR thereof or fragment thereof, e.g., antigen-binding fragment thereof.

2C6 HEAVY CHAIN
(SEQ ID NO: 22)
MELGLSWIFLLAILKGVQCEVQLVESGGGLVQPGRSLRLSCAASGFTE
DDYAMHWVRQAPGKGLEWVSGISWNSGSKGYVDSVKGRFTISRDNAKN
SLYLQMNSLRAEDTALYYCAKDIRIGVAASYYFGMDVWGHGTTVTVSS
(SEQ ID NO: 23)
2C6 CDR-H1: GFTFDDYAMH
(SEQ ID NO: 24)
2C6 CDR-H2: GISWNSGSKGYVDSVKG
(SEQ ID NO: 25)
2C6 CDR-H3: DIRIGVAASYYFGMDV
2C6 LIGHT CHAIN
(SEQ ID NO: 26)
MDMRVPAQLLGLLLLWLPGARCAIQLTQSPSSLSASVGDRVTITCRAS
QGISSVLAWYQQKPGKAPKLLIYDASSLESGVPSRFSGSGSGTDFTLT
ISSLQPEDFATYYCQQFNSYPYTFGQGTKLEIK
(SEQ ID NO: 27)
2C6 CDR-L1: RASQGISSVLA
(SEQ ID NO: 28)
2C6 CDR-L2: DASSLES
(SEQ ID NO: 29)
2C6 CDR-L3: QQFNSYPYT
9H2 HEAVY CHAIN
(SEQ ID NO: 30)
MDWTWRILFLVAAATGAHSQVQLVQSGAEVKKPGASVKVSCKASGYTF
TSYVMHWVRQAPGQRLEWMGWINAGNGNTKYSQKFQGRVTITRDTSAS
TVYMELSSLRSEDTAVYYCARGGMPVAGPGYFYYYGMDVWGQGTTVTV
SS
(SEQ ID NO: 31)
9H2 CDR-H1: GYTFTSYVMH
(SEQ ID NO: 32)
9H2 CDR-H2: WINAGNGNTKYSQKFQG
(SEQ ID NO: 33)
9H2 CDR-H3: GGMPVAGPGYFYYYGMDV
9H2 LIGHT CHAIN
(SEQ ID NO: 34)
METPAQLLFLLLLWLPDTTGEIVLTQSPGTLSLSPGERATLSCRASQS
VSRSYLAWYQQKPGQAPRLLIYGASSRATGIPDRFSGSGSGTDFTLTI
SRLEPEDFAVYCCQQYGSSPWTFGQGTKVEIKRT
(SEQ ID NO: 35)
9H2 CDR-L1: RASQSVSRSYLA
(SEQ ID NO: 36)
9H2 CDR-L2: GASSRAT
(SEQ ID NO: 37)
9H2 CDR-L3: QQYGSSPWT

### Leucovorin/5-Fluorouracil/Sunitinib

The present invention includes combinations including anti-IGF1R antibodies or fragments as discussed herein in association with (i) leucovorin, (ii) sunitinib or (iii) leucovorin and 5-fluorouracil; optionally in further association with a further chemotherapeutic agent; as well as methods of treating colorectal cancer with such combinations.

In an embodiment of the invention, leucovorin, also known as folinic acid and citrovorum factor, is represented by the following structural formula:

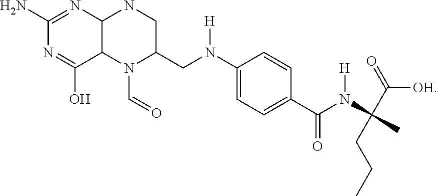

The term includes the salt calcium folinate (or leucovorin calcium). For example, a leucovorin calcium formulation is sold as Wellcovorin® by GlaxoSmithKline.

In an embodiment of the invention, 5-fluorouracil is represented by the following structural formula:

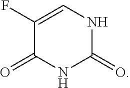

For example, a 5-fluorouracil formulation is sold as Adrucil. In an embodiment of the invention, sunitinib is represented by the following structural formula:

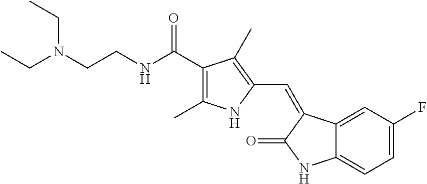

e.g., butanedioic acid thereof. See e.g., U.S. Pat. Nos. 6,573,293; and 7,125,905. For example, a sunitinib malate formulation is sold as Sutent®, by Pfizer, Inc.

### Therapeutic Methods, Dosage and Administration

Methods of the present invention include administration of a therapeutically effective dosage of an IGF1R antibody or antigen-binding fragment thereof of the invention in association with leucovorin and 5-fluorouracil or in association with sunitinib. In an embodiment of the invention, the administration and dosage of leucovorin and 5-fluorouracil or of sunitinib is, when possible, done according to the schedule listed in the product information sheet of the approved agents, in the Physicians' Desk Reference 2003 (Physicians' Desk Reference, 57th Ed); Medical Economics Company; ISBN: 1563634457; 57th edition (November 2002), as well as therapeutic protocols well known in the art.

In an embodiment of the invention, leucovorin is administered intravenously or orally. In general, leucovorin should not be administered intrathecally. 5-fluorouracil is, in an embodiment of the invention, administered intravenously by bolus or infusion. In an embodiment of the invention, sunitinib is administered orally. In an embodiment of the invention, an anti-IGF1R antibody or antigen-binding fragment thereof of the invention is administered parenterally (e.g., intravenous, intraarterially, subcutaneously, intramuscularly or intratumorally).

The scope of the present invention further includes methods for preventing or treating any medical disorder mediated by IGF1R expression or activity or the expression or activity of any ligand of IGF1R such as IGF-1 or IGF-2, for example, osteosarcoma, rhabdomyosarcoma, neuroblastoma, any pediatric cancer, kidney cancer, leukemia, renal transitional cell cancer, Werner-Morrison syndrome, acromegaly, bladder cancer, Wilm's cancer, ovarian cancer, pancreatic cancer, benign prostatic hyperplasia, breast cancer, prostate cancer, bone cancer, lung cancer, gastric cancer, cervical cancer, synovial sarcoma, diarrhea associated with metastatic carcinoid, vasoactive intestinal peptide secreting tumors, gigantism, psoriasis, atherosclerosis, smooth muscle restenosis of blood vessels and inappropriate microvascular proliferation, head and neck cancer, squamous cell carcinoma, multiple myeloma, solitary plasmacytoma, renal cell cancer, retinoblastoma, germ cell tumors, hepatoblastoma, hepatocellular carcinoma, melanoma, rhabdoid tumor of the kidney, Ewing Sarcoma, chondrosarcoma, haemotological malignancy, chronic lymphoblastic leukemia, chronic myelomonocytic leukemia, acute lymphoblastic leukemia, acute lymphocytic leukemia, acute myelogenous leukemia, acute myeloblastic leukemia, chronic myeloblastic leukemia, Hodgekin's disease, non-Hodgekin's lymphoma, chronic lymphocytic leukemia, chronic myelogenous leukemia, myelodysplastic syndrome, hairy cell leukemia, mast cell leukemia, mast cell neoplasm, follicular lymphoma, diffuse large cell lymphoma, mantle cell lymphoma, Burkitt Lymphoma, mycosis fungoides, seary syndrome, cutaneous T-cell lymphoma, chronic myeloproliferative disorders, a central nervous system tumor, brain cancer, glioblastoma, non-glioblastoma brain cancer, meningioma, pituitary adenoma, vestibular schwannoma, a primitive neuroectodermal tumor, medulloblastoma, astrocytoma, anaplastic astrocytoma, oligodendroglioma, ependymoma and choroid plexus papilloma, a myeloproliferative disorder, polycythemia vera, thrombocythemia, idiopathic myelofibrosis, soft tissue sarcoma, thyroid cancer, endometrial cancer, carcinoid cancer, germ cell tumors, liver cancer, gigantism, psoriasis, atherosclerosis, smooth muscle restenosis of blood vessels, inappropriate microvascular proliferation, acromegaly, gigantism, psoriasis, atherosclerosis, smooth muscle restenosis of blood vessels or inappropriate microvascular proliferation, Grave's disease, multiple sclerosis, systemic lupus erythematosus, Hashimoto's Thyroiditis, Myasthenia Gravis, auto-immune thyroiditis and Bechet's disease by administering a therapeutically effective amount of an anti-IGF1R antibody or antigen-binding fragment thereof in association with leucovorin, sunitinib or with leucovorin and 5-fluorouracil; optionally, in further association with a further chemotherapeutic agent (e.g., as discussed herein).

The term colorectal cancer includes all cancers of the colon and/or rectum. For example, the term includes adenocarcinoma of the colon (e.g., mucinous (colloid) adenocarcinoma or signet ring adenocarcinoma). Other types of colorectal cancer included by the term include the following varieties of colon cancer: neuroendocrine, lymphoma, melanoma, squamous cell, sarcoma and carcinoid.

The term colorectal cancer also includes all stages of colorectal cancer; for example, under the Modified Duke Staging System or TNM system (Tumor, Node, Metastisis). The stages associated with these systems are well known by practitioners of ordinary skill in the art.

In an embodiment of the invention, the IGF1R antibody or antigen-binding fragment thereof of the invention in association with leucovorin and 5-fluorouracil or in association with sunitinib is administered to a subject to treat or prevent colorectal cancer wherein the subject is predisposed to colorectal cancer. For example, in an embodiment of the invention, the patient has familial adenomatous polyposis (FAP), hereditary nonpolyposis colon cancer (HNPCC) (i.e., Lynch I Syndrome or Lynch II Syndrome), inflammatory bowel disease, such as chronic ulcerative colitis (UC) or Crohn's disease, other family cancer syndromes (e.g., Peutz-Jegher Syndromem and Familial Juvenile Polyposis), or adenomatous polyps (e.g., sessile (flat with a broad base and no stalk); tubular (composed of tubular glands extending downward from the outer surface of the polyp); villous (composed of fingerlike epithelial projections extending outward from the surface of the bowel mucosa); pedunculated (attached by a narrow base and a long stalk)). In another embodiment of the invention, the subject is not afflicted with any such predisposition.

HNPCC is, in an embodiment of the invention, mediated by one or more genes such as MLH1, MSH2, PMS1, PMS2, and MSH6 and is characterized by an increased risk of several cancers such as colorectal cancer. HNPCC is inherited as an autosomal dominant trait and includes Lynch I syndrome and Lynch II syndrome. In an embodiment of the invention, Lynch I syndrome is characterized by a familial predisposition to colorectal cancer with right-sided predominance and predominantly early-onset proximal colon carcinomas. In an embodiment of the invention, Lynch syndrome II is characterized by a familial predisposition for other primary cancers in addition to the predisposition for colon cancer.

In an embodiment of the invention, familial adenomatous polyposis (FAP) is an inherited condition in which numerous polyps form mainly in the epithelium of the large intestine. In general, while these polyps start out benign, malignant transformation into colon cancer occurs when not treated.

In an embodiment of the invention, inflammatory bowel disease is the name of a group of disorders that cause the intestines to become inflamed (e.g., red and swollen). Typically, ulcerative colitis and crohn's disease are classified as inflammatory bowel diseases. Ulcerative colitis is a form of colitis that includes characteristic ulcers or open sores, in the colon. In an embodiment of the invention, Crohn's disease is a chronic inflammatory disease of the intestines. It primarily causes ulcerations (breaks in the lining) of the small and large intestines, but can affect the digestive system anywhere from the mouth to the anus. Crohn's disease is also called granulomatous enteritis or colitis, regional enteritis, ileitis, or terminal ileitis.

In an embodiment of the invention, Peutz-Jegher's (PJ) syndrome is hereditary condition that results in gastrointestinal polyps and freckles on the skin. The cause for Peutz-Jegher's is an inherited mutation in a gene on chromosome 19, LKB1 or STK 11. The mutation seems to result in a predisposition to benign and cancerous tumors.

In an embodiment of the invention, familial juvenile polyposis (FJP) is an autosomal dominant condition characterized by multiple juvenile polyps of the gastrointestinal (GI) tract. Kindreds have been described in which there is involvement of the colon only, the upper GI tract or both upper and lower GI tracts. FJP is a hamartomatous polyposis syndrome. Although the polyps in PJS are true hamartomata, some may undergo adenomatous change, and these family members are at increased risk for gastrointestinal malignancy. The PJS gene was mapped to chromosome 19p by comparative genomic hybridization and linkage and germline mutations were identified in the serine threonine kinase gene, LKB1.

In an embodiment of the invention, adenomatous polyps (adenomas) of the colon and rectum are benign (noncancerous) growths that may be precursor lesions to colorectal cancer. In general, polyps greater than one centimeter in diameter are associated with a greater risk of cancer. If polyps are not removed, they typically continue to grow and can become cancerous.

The present invention comprises methods for treating or preventing colorectal cancer comprising administering a therapeutically effective amount or dosage of anti-IGF1R or an antigen-binding fragment thereof in association with sunitinib or in association with leucovorin and 5-fluorouracil. The term “therapeutically effective amount” or “therapeutically effective dosage” means that amount or dosage of an antibody or antigen-binding fragment thereof or other therapeutic agent or combination thereof of the invention or composition thereof that will elicit a biological or medical response of a tissue, system, patient, subject or host that is being sought by the administrator (such as a researcher, doctor or veterinarian) which includes any measurable alleviation of the signs, symptoms and/or clinical indicia of colorectal cancer (e.g., tumor growth and/or metastasis) including the prevention, slowing or halting of progression of the colorectal cancer to any degree whatsoever.

In an embodiment of the invention, a therapeutically effective dose of sunitinib is about one 50 mg oral dose taken once daily, e.g., on a schedule of 4 weeks on treatment followed by 2 weeks off, e.g., taken with or without food. In an embodiment of the invention, a therapeutically effective dosage of leucovorin is about 200 mg/m², e.g., by intravenous infusion. In an embodiment of the invention, a therapeutically effective dosage of 5-fluorouracil is about 400 mg/m²-600 mg/m², e.g., by intravenous bolus or infusion.

The anti-IGF1R antibodies and antigen-binding fragments thereof and compositions thereof are, in an embodiment of the invention, administered at a therapeutically effective dosage. For example, in one embodiment of the invention, a “therapeutically effective dosage” of any anti-IGF1R antibody or antigen-binding fragment thereof of the present invention is between about 0.3 and 20 mg/kg of body weight (e.g., about 0.3 mg/kg of body weight, about 0.6 mg/kg of body weight, about 0.9 mg/kg of body weight, about 1 mg/kg of body weight, about 2 mg/kg of body weight, about 3 mg/kg of body weight, about 4 mg/kg of body weight, about 5 mg/kg of body weight, about 6 mg/kg of body weight, about 7 mg/kg of body weight, about 8 mg/kg of body weight, about 9 mg/kg of body weight, about 10 mg/kg of body weight, about 11 mg/kg of body weight, about 12 mg/kg of body weight, about 13 mg/kg of body weight, about 14 mg/kg of body weight, about 15 mg/kg of body weight, about 16 mg/kg of body weight, about 17 mg/kg of body weight, about 18 mg/kg of body weight, about 19 mg/kg of body weight, about 20 mg/kg of body weight), about once per week to about once every 3 weeks (e.g., about once every 1 week or once every 2 weeks or once every 3 weeks).

Dosage regimens may be adjusted to provide the optimum desired response (e.g., a therapeutic response). For example, a single dose may be administered or several divided doses may be administered over time or the dose may be proportionally reduced or increased as indicated by the exigencies or the particular circumstances or requirements of the therapeutic situation. For example, dosage may be determined or adjusted, by a practitioner of ordinary skill in the art (e.g., physician or veterinarian) according to the patient's age, weight, height, past medical history, present medications and the potential for cross-reaction, allergies, sensitivities and adverse side-effects. For example, the physician or veterinarian could start doses of the antibody or antigen-binding fragment of the invention or composition thereof at levels lower than that required in order to achieve the desired therapeutic effect and gradually increase the dosage until the desired effect is achieved. The effectiveness of a given dose or treatment regimen of an antibody or combination of the invention can be determined, for example, by determining whether a tumor being treated in the subject shrinks or ceases to grow. The size and progress of a tumor can be easily determined, for example, by X-ray, magnetic resonance imaging (MRI) or visually in a surgical procedure. In general, tumor size and proliferation can be measured by use of a thymidine PET scan (see e.g., Wells et al., Clin. Oncol. 8: 7-14 (1996)). Generally, the thymidine PET scan includes the injection of a radioactive tracer, such as [2-¹¹C]-thymidine, followed by a PET scan of the patient's body (Vander Borght et al., Gastroenterology 101: 794-799, 1991; Vander Borght et al., J. Radiat. Appl. Instrum. Part A, 42: 103-104 (1991)). Other tracers that can be used include [¹⁸F]-FDG (18-fluorodeoxyglucose), [¹²⁴I]IUdR (5-[124I]iodo-2′-deoxyuridine), [⁷⁶Br]BrdUrd (Bromodeoxyuridine), [¹⁸F]FLT (3′-deoxy-3′ fluorothymidine) or [¹¹C]FMAU (2′-fluoro-5-methyl-1-β-D-arabinofuranosyluracil).

For example, colorectal or colon cancer progress can be monitored, by the physician, by a variety of methods, and the dosing regimen can be altered accordingly. Methods by which to monitor colorectal or colon cancer include CT scan, MRI scan, chest X-ray, PET scan, fecal occult blood tests (FOBTs), flexible proctosigmoidoscopy, total colonoscopy, and barium enema.

The term “subject” or “patient” includes any mammal (e.g., primate, dog, horse, rat, mouse, cat, rabbit) including a human. In an embodiment of the invention, a “subject” or “patient” is an adult human (e.g., 18 years or older) or a human child (e.g., under 18 years of age, for example, less than 1, 1, 2, 3, 4, 5, 6, 7, 8, 9 or 10 years of age); or a female or a male.

### Pharmaceutical Compositions

Methods for treating or preventing colorectal cancer by administering a pharmaceutical composition comprising an anti-IGF1R antibody or antigen-binding fragment thereof of the invention in association with a pharmaceutically acceptable carrier are also within the scope of the present invention (e.g., in a single composition or separately in a kit) as are combinations and compositions including such pharmaceutical compositions. The pharmaceutical compositions may be prepared by any methods well known in the art of pharmacy; see, e.g., Gilman, et al., (eds.) (1990), The Pharmacological Bases of Therapeutics, 8th Ed., Pergamon Press; A. Gennaro (ed.), Remington's Pharmaceutical Sciences, 18th Edition, (1990), Mack Publishing Co., Easton, Pa.; Avis, et al., (eds.) (1993) Pharmaceutical Dosage Forms: Parenteral Medications Dekker, N.Y.; Lieberman, et al., (eds.) (1990) Pharmaceutical Dosage Forms: Tablets Dekker, N.Y.; and Lieberman, et al., (eds.) (1990), Pharmaceutical Dosage Forms: Disperse Systems Dekker, N.Y. In an embodiment of the invention, the antibody or antigen-binding fragment thereof is administered to a subject as part of a pharmaceutical composition comprising sodium acetate (e.g., Trihydrate USP) at 2.30 mg/ml; glacial acetic acid (e.g., USP/Ph. Eur) at 0.18 mg/ml; sucrose (e.g., extra pure NF, Ph. Eur, BP) at 70.0 mg/ml; anti-IGF1R antibody or an antigen-binding fragment thereof at 20.0 mg/ml and water, for example, sterile water (e.g., for injection USP/Ph. Eur); at a pH of about 5.5 to about 6.0 (e.g., 5.5., 5.6, 5.7, 5.8, 5.9, 6.0). If a lyophilized powder thereof (also part of the present invention) is prepared, water is added to reconstitute the composition for use.

A pharmaceutical composition containing an antibody or antigen-binding fragment thereof of the invention, which is optionally in association with a further chemotherapeutic agent, can be prepared using conventional pharmaceutically acceptable excipients and additives and conventional techniques. Such pharmaceutically acceptable excipients and additives include non-toxic compatible fillers, binders, disintegrants, buffers, preservatives, anti-oxidants, lubricants, flavorings, thickeners, coloring agents, emulsifiers and the like. All routes of administration are contemplated including, but not limited to, parenteral (e.g., subcutaneous, intravenous, intraperitoneal, intramuscular, topical, intra-peritoneal, inhalation, intra-cranial) and non-parenteral (e.g., oral, transdermal, intranasal, intraocular, sublingual, rectal and topical).

Injectables can be prepared in conventional forms, either as liquid solutions or suspensions, solid forms suitable for solution or suspension in liquid prior to injection, or as emulsions. The injectables, solutions and emulsions can also contain one or more excipients. Excipients include, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins.

In an embodiment of the invention, pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include sodium chloride injection, Ringers Injection, isotonic dextrose Injection, sterile water injection, dextrose and lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations may be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcellulose, hydroxypropyl methylcellulose and polyvinylpyrrolidone. Emulsifying agents include Polysorbate 80 (TWEEN-80). A sequestering or chelating agent of metal ions includes EDTA (ethylenediaminetetraacetic acid) or EGTA (ethylene glycol tetraacetic acid). Pharmaceutical carriers may also include ethyl alcohol, polyethylene glycol and propylene glycol for water miscible vehicles; and sodium hydroxide, hydrochloric acid, citric acid or lactic acid for pH adjustment.

In an embodiment of the invention, preparations for parenteral administration can include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

The concentration of the antibody or antigen-binding fragment thereof of the invention, which is optionally in association with a further chemotherapeutic agent, can be adjusted so that an injection provides an effective amount to produce the desired pharmacological effect. As discussed herein, the exact dose depends, in part, on the age, weight and condition of the patient or animal as is known in the art.

In an embodiment of the invention, unit-dose parenteral preparations are packaged in an ampoule, a vial or a syringe with a needle. All preparations for parenteral administration must be sterile, as is known and practiced in the art.

In an embodiment of the invention, a sterile, lyophilized powder is prepared by dissolving the antibody or antigen-binding fragment thereof, which is optionally in association with a further chemotherapeutic agent, or a pharmaceutically acceptable derivative thereof, in a suitable solvent. The solvent may contain an excipient which improves the stability or other pharmacological components of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, in one embodiment, about neutral pH. Subsequent sterile filtration of the solution followed by lyophilization under standard conditions known to those of skill in the art provides a desirable formulation. In one embodiment, the resulting solution will be apportioned into vials for lyophilization. Each vial can contain a single dosage or multiple dosages of the anti-IGF1R antibody or antigen-binding fragment thereof or composition thereof. Overfilling vials with a small amount above that needed for a dose or set of doses (e.g., about 10%) is acceptable so as to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In an embodiment of the invention, for reconstitution, the lyophilized powder is added to sterile water or other liquid suitable carrier. The precise amount depends upon the selected therapy being given. Such amount can be empirically determined.

Administration by inhalation can be provided by using, e.g., an aerosol containing sorbitan trioleate or oleic acid, for example, together with trichlorofluoromethane, dichlorofluoromethane, dichlorotetrafluoroethane or any other biologically compatible propellant gas; it is also possible to use a system containing an IGF1R inhibitor, which is optionally in association with a further chemotherapeutic agent, by itself or associated with an excipient, in powder form.

Implantation of a slow-release or sustained-release system, such that a constant level of dosage is maintained, is also contemplated herein. Briefly, an active agent (e.g., anti-IGF1R, which is optionally in association with a further chemotherapeutic agent) is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, or ethylene/vinyloxyethanol copolymer, that is insoluble in body fluids. The antibody or fragment diffuses through the outer polymeric membrane in a release rate controlling step. The percentage of active agent contained in such parenteral compositions is highly dependent on the specific nature thereof, as well as the activity of the antibody or antigen-binding fragment, which is optionally in association with a further chemotherapeutic agent, and the needs of the subject.

Agents set forth herein can be formulated into a sustained release formulation including liposomal formulations such as unilamellar vesicular (ULV) and multilamellar vesicular (MLV) liposomes and DepoFoam™ particles (Kim et al., Biochim. Biophys. Acta (1983) 728(3):339-348; Kim, Methods Neurosci. (1994) 21: 118-131; Kim et al., Anesthesiology (1996) 85(2): 331-338; Katre et al., J. Pharm. Sci. (1998) 87(11): 1341-1346). A feature of the DepoFoam system is that, inside each DepoFoam particle, discontinuous internal aqueous chambers, bounded by a continuous, non-concentric network of lipid membranes render a higher aqueous volume-to-lipid ratio and much larger particle diameters compared with MLV.

In an embodiment of the invention, sunitinib is sunitinib malate, and, in an embodiment of the invention, is supplied in capsules containing sunitinib malate equivalent to 12.5 mg, 25 mg or 50 mg of sunitinib together with mannitol, croscarmellose sodium, povidone (K-25) and magnesium stearate as inactive ingredients.

In an embodiment of the invention, leucovorin is leucovorin calcium, and, in an embodiment of the invention, is in a tablet containing 5 mg of leucovorin (equivalent to 5.40 mg of anhydrous leucovorin calcium) and the following inactive ingredients: corn starch, dibasic calcium phosphate, magnesium stearate, and pregelatinized starch; or 15 mg of leucovorin (equivalent to 16.20 mg of anhydrous leucovorin calcium) and the following inactive ingredients: lactose, magnesium stearate, microcrystalline cellulose, pregelatinized starch, and sodium starch glycolate.

In an embodiment of the invention, 5-fluorouracil is a colorless to faint yellow aqueous, sterile, nonpyrogenic injectable solution for intravenous administration wherein each 10 mL contains 500 mg of fluorouracil; and wherein pH is adjusted to 8.6 to 9.4 with sodium hydroxide and hydrochloric acid if necessary.

### EXAMPLES

The present invention is intended to exemplify the present invention and not to be a limitation thereof. Methods and compositions disclosed below fall within the scope of the present invention.

### Example 1

### Inhibition of Colorectal Tumor Growth in Xenograft Models

In this example, the efficacy of treatment regimens, comprising anti-IGF1R in association with sunitinib or in association with 5-fluorouracil and leucovorin, for treating colorectal tumor growth in xengraft models was demonstrated.

In these experiments, 5 million WiDr human colorectal adenocarcinoma cells, mixed 1:1 with Matrigel, were inoculated subcutaneously to the flank of each nude mouse (nu/nu). Dosing of anti-IGF1R and/or the second chemotherapeutic agent (sunitinib or leucovorin/5-fluorouracil) was initiated when the tumors reached an average size of 100 mm³. The vehicle used for anti-IGF1R antibody, leucovorin, 5-FU and sunitinib was saline (0.9% NaCl).

The treatment regimen followed for mice treated with anti-IGF1R and 5-fluorouracil and leucovorin is set forth in Table 1.

TABLE 1
Combination efficacy study design in WiDr

| | | Number | | |
|---|---|---|---|---|
| | Mouse | and Sex | | |
| Groups | strain | of Mice | Dosing Schedule | Route |
| Control IgG1 | nude | 10 female | 2x/week | IP\hat{ } |
| 0.5 mg anti-IGF1R* | nude | 10 female | 2x/week | IP |
| 60 mpk 5-FU^{#}/10 mpk | nude | 10 female | 2x/week | IP |
| Leucovorin | | | | |
| 0.5 mg anti-IGF1R + | nude | 10 female | 2x/week | IP |
| 5-FU/Leucovorin | | | | |

The results of these experiments are set forth below in Table 2.

TABLE 2
Summary of combination efficacy in WiDr

| | % |
|---|---|
| | Growth |
| Groups | Inhibition |
| Control IgG1 | 0 |
| 0.5 mg anti-IGF1R | 30 |
| 60 mpk 5-FU/10 mpk Leucovorin | 44^{a} |
| 0.5 mg anti-IGF1R + 5-FU/Leucovorin | 67^{b} |

^{a}indicates the treated group is significantly better than the control group (p < 0.05).
^{b}indicates the combination treated group is significantly better than either single agent used alone (p < 0.05).

The average size (and standard error of the mean) of the tumors of each treatment group over time are summarized below in Table 3.

TABLE 3
Summary of combination efficacy in WiDr

| | | | | | | | | |
|---|---|---|---|---|---|---|---|---|
| Day (post inoculation) | 10 | 14 | 17 | 21 | 24 | 28 | 31 | 35 |

Mean tumor volume (mm³)

| | | | | | | | | |
|---|---|---|---|---|---|---|---|---|
| Control IgG1 0.5 mg + Vehicle | 102.8 | 198.7 | 279.0 | 366.6 | 458.5 | 571.9 | 681.5 | 797.8 |
| 0.5 mg anti-IGF1R | 93.1 | 148.0 | 192.0 | 254.1 | 317.0 | 403.1 | 474.5 | 577.6 |
| 10 mpk Leucovorin/60 mpk 5-FU | 95.4 | 171.7 | 217.6 | 261.6 | 295.0 | 341.5 | 407.7 | 481.7 |
| 0.5 mg anti-IGF1R + 10 mpk | 93.4 | 136.9 | 161.3 | 180.8 | 191.9 | 239.6 | 269.3 | 322.5 |
| Leucovorin/60 mpk 5-FU | | | | | | | | |

Standard error of the mean

| | | | | | | | | |
|---|---|---|---|---|---|---|---|---|
| Control IgG1 0.5 mg + Vehicle | 4.5 | 15.3 | 17.0 | 23.3 | 35.3 | 47.5 | 70.5 | 114.6 |
| 0.5 mg anti-IGF1R | 4.1 | 11.3 | 17.8 | 24.3 | 29.5 | 41.9 | 48.0 | 62.0 |
| 10 mpk Leucovorin/60 mpk 5-FU | 3.2 | 11.6 | 13.7 | 19.0 | 20.2 | 28.9 | 40.7 | 50.9 |
| 0.5 mg anti-IGF1R + 10 mpk | 3.8 | 12.7 | 18.0 | 26.2 | 28.6 | 41.7 | 48.8 | 61.7 |
| Leucovorin/60 mpk 5-FU | | | | | | | | |

The treatment regimen followed for mice treated with anti-IGF1R and sunitinib is set forth in Table 4.

TABLE 4
Combination efficacy study design in WiDr

| | | Number | | |
|---|---|---|---|---|
| | Mouse | and Sex | | |
| Groups | strain | of Mice | Dosing Schedule | Route |
| Control IgG1 | nude | 10 female | 2x/week | IP |
| 0.5 mg anti-IGF1R | nude | 10 female | 2x/week | IP |
| 40 mpk Sunitinib | nude | 10 female | QD^{£} | PO^{±} |
| 0.5 mg anti-IGF1R + | nude | 10 female | QD Sunitinib | PO, IP |
| 40 mpk Sunitinib | | | 2x/week anti- | |
| | | | IGF1R | |

The results of these experiments are set forth below in Table 5.

TABLE 5
Summary of combination efficacy in WiDr

| | % |
|---|---|
| | Growth |
| Groups | Inhibition |
| Control IgG1 | 0 |
| 0.5 mg anti-IGF1R | 30 |
| 40 mpk Sunitinib | 79^{a} |
| 0.5 mg anti-IGF1R + 40 mpk Sunitinib | 90^{b} |

^{a}indicates the treated group is significantly better than the control group (p < 0.05).
^{b}indicates the combination treated group is significantly better than either single agent used alone (p < 0.05).

The average size (and standard error of the mean) of the tumors of each treatment group over time are summarized below in Table 6.

TABLE 6
Summary of combination efficacy in WiDr

| | | | | | | | | |
|---|---|---|---|---|---|---|---|---|
| Day (post inoculation) | 10 | 14 | 17 | 21 | 24 | 28 | 31 | 35 |

Mean tumor volume (mm³)

| | | | | | | | | |
|---|---|---|---|---|---|---|---|---|
| Control IgG1 0.5 mg + Vehicle | 102.8 | 198.7 | 279.0 | 366.6 | 458.5 | 571.9 | 681.5 | 797.8 |
| 0.5 mg anti-IGF1R | 93.1 | 148.0 | 192.0 | 254.1 | 317.0 | 403.1 | 474.5 | 577.6 |
| 40 mpk Sunitinib | 95.8 | 125.4 | 143.1 | 175.6 | 194.8 | 205.9 | 216.0 | 238.6 |
| 0.5 mg anti-IGF1R + 40 mpk Sunitinib | 96.2 | 110.1 | 121.0 | 132.4 | 141.4 | 153.3 | 153.5 | 167.1 |

Standard error of the mean

| | | | | | | | | |
|---|---|---|---|---|---|---|---|---|
| Control IgG1 0.5 mg + Vehicle | 4.5 | 15.3 | 17.0 | 23.3 | 35.3 | 47.5 | 70.5 | 114.6 |
| 0.5 mg anti-IGF1R | 4.1 | 11.3 | 17.8 | 24.3 | 29.5 | 41.9 | 48.0 | 62.0 |
| 40 mpk Sunitinib | 4.3 | 16.7 | 19.4 | 29.7 | 30.6 | 29.2 | 30.0 | 33.2 |
| 0.5 mg anti-IGF1 R + 40 mpk Sunitinib | 3.8 | 7.9 | 9.7 | 12.4 | 13.5 | 15.8 | 17.0 | 20.2 |

* anti-IGF1R = LCF/HCA (γ1, κ)
^{#} 5-FU = 5-fluorouracil
^{\hat{ }} IP = administered by intraperitoneal injection
^{±} PO = per os administration (orally)
^{£} QD = once a day (dosing schedule)

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

We claim:

1. A method for treating or preventing colorectal cancer in a subject comprising administering a therapeutically effective amount an isolated antibody or antigen-binding fragment thereof comprising one or more members selected from the group consisting of:
(a) CDR-L1, CDR-L2 and CDR-L3 of the variable region of light chain C, light chain D, light chain E or light chain F; or
(b) CDR-H1, CDR-H2 and CDR-H3 of the variable region of heavy chain A or heavy chain B; or both; in association with
leucovorin and 5-fluorouracil; or in association with sunitinib.

2. The method of claim 1 wherein:
CDR-L1 comprises the amino acid sequence:
(SEQ ID NO: 1)
Arg Ala Ser Gln Ser Ile Gly Ser Ser Leu His;
CDR-L2 comprises the amino acid sequence:

| | |
|---|---|
| Tyr Ala Ser Gln Ser Leu Ser; | (SEQ ID NO: 2) |

CDR-L3 comprises the amino acid sequence:

| | |
|---|---|
| His Gln Ser Ser Arg Leu Pro His Thr; | (SEQ ID NO: 3) |

CDR-H1 comprises the amino acid sequence:
(SEQ ID NO: 4)
Ser Phe Ala Met His
or
(SEQ ID NO: 5)
Gly Phe Thr Phe Ser Ser Phe Ala Met His;
CDR-H2 comprises the amino acid sequence:
(SEQ ID NO: 6)
Val Ile Asp Thr Arg Gly Ala Thr Tyr Tyr Ala Asp
Ser Val Lys Gly;
and
CDR-H3 comprises the amino acid sequence:
(SEQ ID NO: 7)
Leu Gly Asn Phe Tyr Tyr Gly Met Asp Val.

3. The method of claim 1 wherein the antibody or fragment comprises a light chain variable region comprising amino acids 20-128 of SEQ ID NO: 9, 11, 13 or 15 and a heavy chain variable region comprising amino acids 20-137 of SEQ ID NO: 17 or 19.

4. The method of claim 1 wherein said antibody or antigen-binding fragment is an antibody which is a monoclonal antibody.

5. The method of claim 1 wherein said antibody or fragment is an antibody and the antibody is a labeled antibody, bivalent antibody, a polyclonal antibody, a bispecific antibody, a chimeric antibody, a recombinant antibody, an anti-idiotypic antibody, a humanized antibody or a bispecific antibody.

6. The method of claim 1 wherein the antibody or fragment is a fragment and the fragment is a camelized single domain antibody, a diabody, an scfv, an scfv dimer, a dsfv, a (dsfv)₂, a dsFv-dsfv′, a bispecific ds diabody, a nanobody, an Fv, an Fab, an Fab′, an F(ab′)₂, or a domain antibody.

7. The method of claim 1 wherein the antibody or fragment is linked to a constant region.

8. The method of claim 7 wherein the constant region is a κ light chain, γ1 heavy chain, γ2 heavy chain, γ3 heavy chain or γ4 heavy chain.

9. The method of claim 1 wherein the subject is administered a further chemotherapeutic agent or an anti-cancer therapeutic procedure.

10. The method of claim 9 wherein the anti-cancer therapeutic procedure is anti-cancer radiation therapy or surgical tumorectomy.

11. The method of claim 9 wherein the further chemotherapeutic agent is an anti-cancer chemotherapeutic agent.

12. The method of claim 9 wherein the further chemotherapeutic agent is one or more members selected from the group consisting of:
everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a BcI-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380, 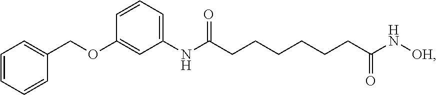
CG-781, CG-1521, 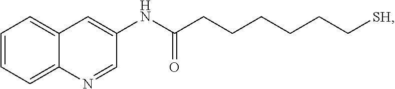
SB-556629, chlamydocin, JNJ-16241199, 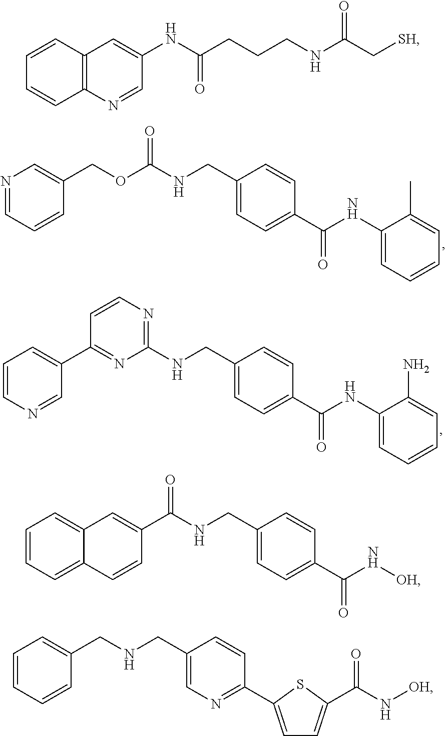
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258, 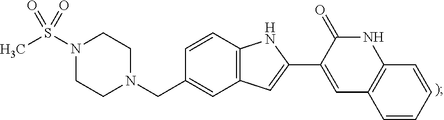
3-[5-(methylsulfonylpiperadinemethyl)-indolyl]-quinolone, vatalanib, AG-013736, AVE-0005, the acetate salt of [D-Ser(Bu t)6,Azgly 10](pyro-Glu-His-Trp-Ser-Tyr-D-Ser(Bu t)-Leu-Arg-Pro-Azgly-NH₂ acetate[C₅₉H₈₄N₁₈O₁₄—(C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 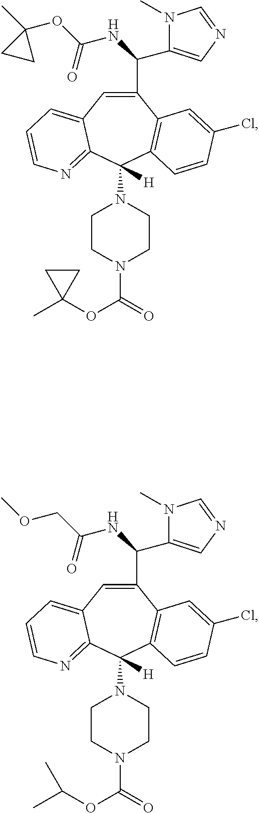
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.

13. The method of claim 1 wherein the antibody or fragment, the leucovorin, the 5-fluorouracil and the sunitinib are in separate pharmaceutical compositions, each independently further comprising a pharmaceutically acceptable carrier.

14. The method of claim 1 wherein the subject is a human.

15. The method of claim 1 wherein the subject suffers from one or more conditions selected from the group consisting of: familial adenomatous polyposis, hereditary nonpolyposis colon cancer, Lynch I Syndrome, Lynch II Syndrome, inflammatory bowel disease, chronic ulcerative colitis (UC), Crohn's disease, a family cancer syndrome, Peutz-Jegher Syndrome, familial juvenile polyposis and one or more adenomatous polyps.

16. A combination comprising an isolated antibody or antigen-binding fragment thereof comprising one or more members selected from the group consisting of:
(a) CDR-L1, CDR-L2 and CDR-L3 of the variable region of light chain C, light chain D, light chain E or light chain F; or
(b) CDR-H1, CDR-H2 and CDR-H3 of the variable region of heavy chain A or heavy chain B; or both; in association with
(i) leucovorin and 5-fluorouracil;
(ii) leucovorin; or
(iii) sunitinib; optionally in further association with a further chemotherapeutic agent.

17. The combination of claim 16 wherein the antibody or fragment comprises a light chain variable region comprising amino acids 20-128 of SEQ ID NO: 9, 11, 13 or 15 and/or a heavy chain variable region comprising amino acids 20-137 of SEQ ID NO: 17 or 19.

18. The combination of claim 16 wherein the further chemotherapeutic agent is one or more members selected from the group consisting of:
everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a BcI-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR, KRX-0402, lucanthone, LY 317615, neuradiab, vitespan, Rta 744, Sdx 102, talampanel, atrasentan, Xr 311, romidepsin, ADS-100380, 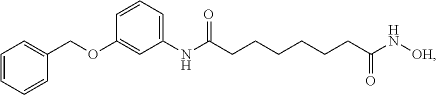
CG-781, CG-1521, 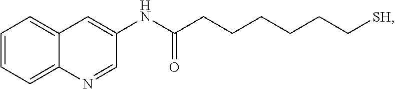
SB-556629, chlamydocin, JNJ-16241199, 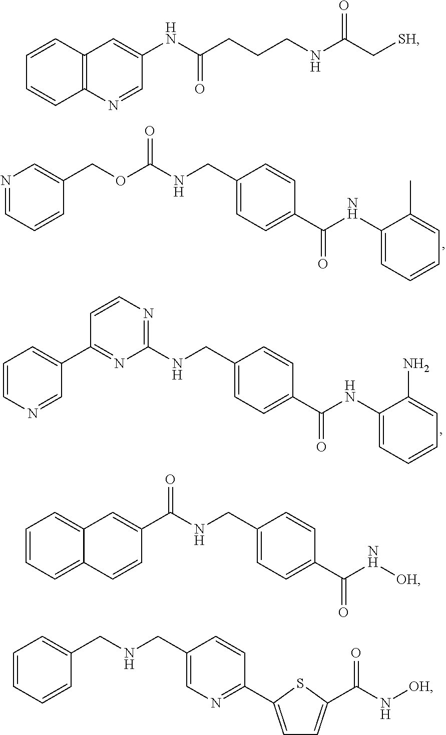
vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, irinotecan; PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258, 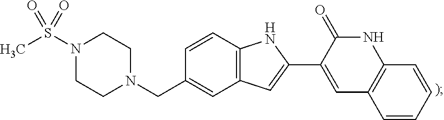
3-[5-(methylsulfonylpiperadinemethyl)-indolyl]-quinolone, vatalanib, AG-013736, AVE-0005, the acetate salt of [D-Ser(Bu t)6,Azgly 10](pyro-Glu-His-Trp-Ser-Tyr-D-Ser(Bu t)-Leu-Arg-Pro-Azgly-NH₂ acetate[C₅₉H₈₄N₁₈O₁₄ (C₂H₄O₂)ₓ where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafarnib, 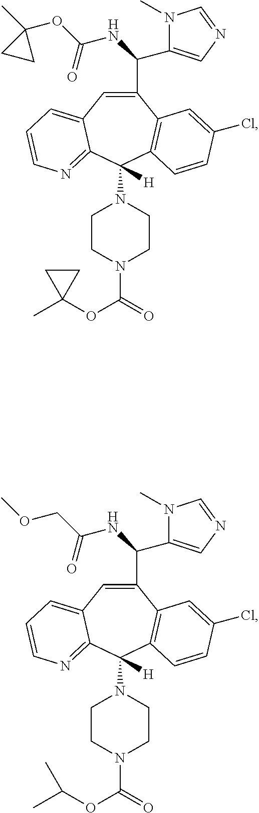
BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyclophosphamide, cyproterone, cytarabine, dacarbazine, dactinomycin, daunorubicin, diethylstilbestrol, epirubicin, fludarabine, fludrocortisone, fluoxymesterone, flutamide, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mercaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa and darbepoetin alfa.


SEQUENCE LISTING (37 sequences)

SEQ ID NO: 1 - protein, 11 aa, Homo sapiens
RASQSIGSSLH

SEQ ID NO: 2 - protein, 7 aa, Homo sapiens
YASQSLS

SEQ ID NO: 3 - protein, 9 aa, Homo sapiens
HQSSRLPHT

SEQ ID NO: 4 - protein, 5 aa, Homo sapiens
SFAMH

SEQ ID NO: 5 - protein, 10 aa, Homo sapiens
GFTFSSFAMH

SEQ ID NO: 6 - protein, 16 aa, Homo sapiens
VIDTRGATYYADSVKG

SEQ ID NO: 7 - protein, 10 aa, Homo sapiens
LGNFYYGMDV

SEQ ID NO: 8 - DNA, 384 bp, Homo sapiens
  CDS (1)..(384)
atgtcgccatcacaactcattgggtttctgctgctctgggttccagcctccaggggtgaaattgtgctgactcagagcccagactctctgtctgtgactccaggcgagagagtcaccatcacctgccgggccagtcagagcattggtagtagcttacactggtaccagcagaaaccaggtcagtctccaaagcttctcatcaagtatgcatcccagtccctctcaggggtcccctcgaggttcagtggcagtggatctgggacagatttcaccctcaccatcagtagcctcgaggctgaagatgctgcagcgtattactgtcatcagagtagtcgtttacctcacactttcggccaagggaccaaggtggagatcaaacgtacg

SEQ ID NO: 9 - protein, 128 aa, Homo sapiens
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDAAAYYCHQSSRLPHTFGQGTKVEIKRT

SEQ ID NO: 10 - DNA, 384 bp, Homo sapiens
  CDS (1)..(384)
atgtcgccatcacaactcattgggtttctgctgctctgggttccagcctccaggggtgaaattgtgctgactcagagcccagactctctgtctgtgactccaggcgagagagtcaccatcacctgccgggccagtcagagcattggtagtagcttacactggtaccagcagaaaccaggtcagtctccaaagcttctcatcaagtatgcatcccagtccctctcaggggtcccctcgaggttcagtggcagtggatctgggacagatttcaccctcaccatcagtagcctcgaggctgaagatttcgcagtgtattactgtcatcagagtagtcgtttacctcacactttcggccaagggaccaaggtggagatcaaacgtacg

SEQ ID NO: 11 - protein, 128 aa, Homo sapiens
MSPSQLIGFLLLWVPASRGEIVLTQSPDSLSVTPGERVTITCRASQSIGSSLHWYQQKPGQSPKLLIKYASQSLSGVPSRFSGSGSGTDFTLTISSLEAEDFAVYYCHQSSRLPHTFGQGTKVEIKRT

SEQ ID NO: 12 - DNA, 384 bp, Homo sapiens
  CDS (1)..(384)
atgtcgccatcacaactcattgggtttctgctgctctgggttccagcctccaggggtgaaattgtgctgactcagagcccaggtaccctgtctgtgtctccaggcgagagagccaccctctcctgccgggccagtcagagcattggtagtagcttacactggtaccagcagaaaccaggtcaggctccaaggcttctcatcaagtatgcatcccagtccctctcagggatccccgataggttcagtggcagtggatctgggacagatttcaccctcaccatcagtagactggagcctgaagatgctgcagcgtattactgtcatcagagtagtcgtttacctcacactttcggccaagggaccaaggtggagatcaaacgtaca

SEQ ID NO: 13 - protein, 128 aa, Homo sapiens
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDAAAYYCHQSSRLPHTFGQGTKVEIKRT

SEQ ID NO: 14 - DNA, 384 bp, Homo sapiens
  CDS (1)..(384)
atgtcgccatcacaactcattgggtttctgctgctctgggttccagcctccaggggtgaaattgtgctgactcagagcccaggtaccctgtctgtgtctccaggcgagagagccaccctctcctgccgggccagtcagagcattggtagtagcttacactggtaccagcagaaaccaggtcaggctccaaggcttctcatcaagtatgcatcccagtccctctcagggatccccgataggttcagtggcagtggatctgggacagatttcaccctcaccatcagtagactggagcctgaagatttcgcagtgtattactgtcatcagagtagtcgtttacctcacactttcggccaagggaccaaggtggagatcaaacgtaca

SEQ ID NO: 15 - protein, 128 aa, Homo sapiens
MSPSQLIGFLLLWVPASRGEIVLTQSPGTLSVSPGERATLSCRASQSIGSSLHWYQQKPGQAPRLLIKYASQSLSGIPDRFSGSGSGTDFTLTISRLEPEDFAVYYCHQSSRLPHTFGQGTKVEIKRT

SEQ ID NO: 16 - DNA, 411 bp, Homo sapiens
  CDS (1)..(411)
atggagtttgggctgagctgggttttccttgttgctatattaaaaggtgtccagtgtgaggttcagctggtgcagtctgggggaggcttggtaaagcctggggggtccctgagactctcctgtgcagcctctggattcaccttcagtagctttgctatgcactgggttcgccaggctccaggaaaaggtctggagtggatatcagttattgatactcgtggtgccacatactatgcagactccgtgaagggccgattcaccatctccagagacaatgccaagaactccttgtatcttcaaatgaacagcctgagagccgaggacactgctgtgtattactgtgcaagactggggaacttctactacggtatggacgtctggggccaagggaccacggtcaccgtctcctca

SEQ ID NO: 17 - protein, 137 aa, Homo sapiens
MEFGLSWVFLVAILKGVQCEVQLVQSGGGLVKPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 18 - DNA, 411 bp, Homo sapiens
  CDS (1)..(411)
atggagtttgggctgagctgggttttccttgttgctatattaaaaggtgtccagtgtgaggttcagctggtgcagtctgggggaggcttggtacagcccggggggtccctgagactctcctgtgcagcctctggattcaccttcagtagctttgctatgcactgggttcgccaggctccaggaaaaggtctggagtggatatcagttattgatactcgtggtgccacatactatgcagactccgtgaagggccgattcaccatctccagagacaatgccaagaactccttgtatcttcaaatgaacagcctgagagccgaggacactgctgtgtattactgtgcaagactggggaacttctactacggtatggacgtctggggccaagggaccacggtcaccgtctcctca

SEQ ID NO: 19 - protein, 137 aa, Homo sapiens
MEFGLSWVFLVAILKGVQCEVQLVQSGGGLVQPGGSLRLSCAASGFTFSSFAMHWVRQAPGKGLEWISVIDTRGATYYADSVKGRFTISRDNAKNSLYLQMNSLRAEDTAVYYCARLGNFYYGMDVWGQGTTVTVSS

SEQ ID NO: 20 - protein, 123 aa, Homo sapiens
VQLLESGGGLVQPGGSLRLSCTASGFTFSSYAMNWVRQAPGKGLEWVSAISGSGGTTFYADSVKGRFTISRDNSRTTYLQMNSLRAEDTAVYYCAKDLGWSDSYYYYYGMDVWGQGTTVTVSS

SEQ ID NO: 21 - protein, 107 aa, Homo sapiens [sequence omitted]

SEQ ID NO: 22 - protein, 144 aa, Homo sapiens
MELGLSWIFLLAILKGVQCEVQLVESGGGLVQPGRSLRLSCAASGFTFDDYAMHWVRQAPGKGLEWVSGISWNSGSKGYVDSVKGRFTISRDNAKNSLYLQMNSLRAEDTALYYCAKDIRIGVAASYYFGMDVWGHGTTVTVSS

SEQ ID NO: 23 - protein, 10 aa, Homo sapiens
GFTFDDYAMH

SEQ ID NO: 24 - protein, 17 aa, Homo sapiens
GISWNSGSKGYVDSVKG

SEQ ID NO: 25 - protein, 16 aa, Homo sapiens
DIRIGVAASYYFGMDV

SEQ ID NO: 26 - protein, 129 aa, Homo sapiens
MDMRVPAQLLGLLLLWLPGARCAIQLTQSPSSLSASVGDRVTITCRASQGISSVLAWYQQKPGKAPKLLIYDASSLESGVPSRFSGSGSGTDFTLTISSLQPEDFATYYCQQFNSYPYTFGQGTKLEIK

SEQ ID NO: 27 - protein, 11 aa, Homo sapiens
RASQGISSVLA

SEQ ID NO: 28 - protein, 7 aa, Homo sapiens
DASSLES

SEQ ID NO: 29 - protein, 9 aa, Homo sapiens
QQFNSYPYT

SEQ ID NO: 30 - protein, 146 aa, Homo sapiens
MDWTWRILFLVAAATGAHSQVQLVQSGAEVKKPGASVKVSCKASGYTFTSYVMHWVRQAPGQRLEWMGWINAGNGNTKYSQKFQGRVTITRDTSASTVYMELSSLRSEDTAVYYCARGGMPVAGPGYFYYYGMDVWGQGTTVTVSS

SEQ ID NO: 31 - protein, 10 aa, Homo sapiens
GYTFTSYVMH

SEQ ID NO: 32 - protein, 17 aa, Homo sapiens
WINAGNGNTKYSQKFQG

SEQ ID NO: 33 - protein, 18 aa, Homo sapiens
GGMPVAGPGYFYYYGMDV

SEQ ID NO: 34 - protein, 130 aa, Homo sapiens
METPAQLLFLLLLWLPDTTGEIVLTQSPGTLSLSPGERATLSCRASQSVSRSYLAWYQQKPGQAPRLLIYGASSRATGIPDRFSGSGSGTDFTLTISRLEPEDFAVYCCQQYGSSPWTFGQGTKVEIKRT

SEQ ID NO: 35 - protein, 12 aa, Homo sapiens
RASQSVSRSYLA

SEQ ID NO: 36 - protein, 7 aa, Homo sapiens
GASSRAT

SEQ ID NO: 37 - protein, 9 aa, Homo sapiens
QQYGSSPWT